(19) United States
(12) Patent Application Publication
(10) Pub. No.: US 2004/0176372 A1
(43) Pub. Date: Sep. 9, 2004

(54) Pin1-modulating compounds and methods of use thereof

(75) Inventors: Robert K. Suto, Maynard, MA (US); Timothy D. McKee, Waltham, MA (US)

Correspondence Address: LAHIVE & COCKFIELD, LLP., 28 STATE STREET, BOSTON, MA, 02109, US

(73) Assignee: Pintex Pharmaceuticals, Inc., Watertown, MA

(21) Appl. No.: 10/379,377

(22) Filed: Mar. 3, 2003

Related U.S. Application Data
(60) Provisional application No. 60/361,206.

(51) Int. Cl. A61K 31/5377; A61K 31/4439; A61K 31/454; A61K 31/4745; A61K 31/433; A61K 31/4245

(52) U.S. Cl. 514/235.5; 514/291; 514/341; 514/369; 514/394; 514/406; 514/364; 514/326

(57) ABSTRACT

The invention is directed to modulators, e.g, inhibitors, of Pin1 and Pin1-related proteins and the use of such modulators for treatment of Pin1 associated states, e.g., for the treatment of cancer.

### RELATED APPLICATIONS

This application claims priority to U.S. Provisional Application No. 60/361,206 filed Mar. 1, 2002, entitled “Pin1-Modulating Compounds and Methods of Use Thereof.”

This application is related to U.S. Provisional Application Serial No. 60/361,246, filed Mar. 1, 2002, entitled “Pin1-Modulating Compounds and Methods of Use Thereof”; U.S. Provisional Application Serial No: 60/361,231, filed Mar. 1, 2002, entitled “Pin1-Modulating Compounds and Methods of Use Thereof”; U.S. Provisional Application Serial No. 60/361,227, filed on Mar. 1, 2002; entitled “Methods for Designing Specific Inhibitors for Pin1 Proline Isomerase and Pin1-Related Molecules”; U.S. Provisional Application No. 60/360,799 filed Mar. 1, 2002, entitled “Methods of Treating Pin1 Associated Disorders”; U.S. Provisional Application Serial No. 60/414,077, filed Sep. 26, 2002, entitled “Pin1-Modulating Compounds and Methods of Use Thereof; PTZ-034-2, entitled “Pin1-Modulating Compounds and Methods of Use Thereof”, filed Mar. 3, 2003; PTZ-034, entitled “Pin1-Modulating Compounds and Methods of Use Thereof”, filed Mar. 3, 2003; PTZ-035, entitled entitled “Pin1-Modulating Compounds and Methods of Use Thereof,” filed Mar. 3, 2003; PTZ-036-2, entitled “Pin1-Modulating Compounds and Methods of Use Thereof”, filed Mar. 3, 2003; PTZ-036, entitled “Pin1-Modulating Compounds and Methods of Use Thereof”, filed Mar. 3, 2003; PTZ-037, entitled “Methods of Treating Pin1 Associated Disorders,” filed Mar. 3, 2003; PTZ-009, entitled “Methods for Designing Specific Inhibitors for Pin1 Proline Isomerase and Pin1-Related Molecules,” filed Mar. 3, 2003; PTZ-046-2, entitled “Pin1-Modulating Compounds and Methods of Use Thereof”, filed Mar. 3, 2003; and PTZ-046, entitled “Pin1-Modulating Compounds and Methods of Use Thereof”, filed Mar. 3, 2003. The entire contents of each of the aforementioned applications are hereby expressly incorporated herein by reference in their entireties.

### BACKGROUND OF THE INVENTION

The peptidyl-prolyl cis-trans isomerases (PPIases), or rotamases, are a family of ubiquitous enzymes that catalyze the cis/trans isomerization of the peptide bond on the N-terminal side of proline residues in proteins (Hunter, Cell 92:141-142, 1998). PPIases are divided into three classes, cyclophilins (Cyps), FK-506 binding proteins (FKBPs) and the Pin1/parvulin class.

Cyclophilins and FKBPs are distinguished by their ability to bind the clinically immunosuppressive drugs cyclosporin and FK506, respectively (Schreiber, Science 251:283-7, 1991; Hunter, supra). Upon binding of these drugs, there are two common outcomes: inhibition of the PPIase activity and inhibition of the common target calcineurin. The inhibition of calcineurin phosphatase activity prevents lymphocytes from responding to antigen-induced mitogenic signals, thus resulting in immunusuppression. However, the inhibition of the PPIase activity is apparently unrelated to the immunosuppressive property of the drug/PPIase complexes. Even more surprisingly, deletion of all 8 known cyclophilins and 4 FKBPs in the same cells does not result in any significant phenotype (Dolinski et al., Proc. Natl. Acad. Sci. USA 94:13093-131098, 1997).

In contrast, members of the Pin1/parvulin class of PPIases bind neither of these immunosuppressive drugs, and are structurally unrelated to the other two classes of PPIases. Known members of the Pin1/parvulin class include Pins 1-3 (Lu et al., Nature 380;544-547, 1996), Pin-L (Campbell et al., Genomics 44:157-162, 1997), parvulin (Rahfeld, et al., Proc. Natl. Acad. Sci. USA 93:447-451, 1996) and Ess1/Pft1 (Hanes et al., Yeast 5:55-72, 1989; and Hani, et al. FEBS Letts 365:198-202, 1995).

Pin1 is a highly conserved protein that catalyzes the isomerization of only phosphorylated Ser/Thr-Pro bonds (Rananathan, R. et al. (1997) Cell 89:875-86; Yaffe, et al. 1997, Science 278:1957-1960; Shen, et al. 1998,Genes Dev. 12:706-720; Lu, et al. 1999, Science 283:1325-1328; Crenshaw, et al. 1998, Embo J. 17:1315-1327; Lu, et al. 1999, Nature 399:784-788; Zhou, et al. 1999, Cell Mol. Life Sci. 56:788-806). In addition, Pin1 contains an N-terminal WW domain, which functions as a phosphorylated Ser/Thre-Pro binding module (Sudol, M. (1996) Prog. Biophys. Mol Biol 65:113-32). This phosphorylation-dependent interaction targets Pin1 to a subset of phosphorylated substrates, including Cdc25, Wee 1, Myt1, Tau-Rad4, and the C-terminal domain of RNA polymerase II large domain (Crenshaw, D. G., et al. (1998) Embo. J 17:1315-27; Shen, M. (1998) Genes Dev. 12:706-20; Wells, N. J. (1999) J Cell. Sci. 112: 3861-71).

The specificity of Pin1 activity is essential for cell growth; depletion or mutations of Pin1 cause growth arrest, affect cell cycle checkpoints and induce premature mitotic entry, mitotic arrest and apoptosis in human tumor cells, yeast or Xenopus extracts (Lu, et al. 1996, Nature 380:544-547; Winkler, et al. 200, Science 287:1644-1647; Hani, et al. 1999. J. Biol. Chem. 274:108-116). In addition, Pin1 is dramatically overexpressed in human cancer samples and the levels of Pin1 are correlated with the aggressiveness of tumors. Moreover, inhibition of Pin1 by various approaches, including Pin1 antisense polynucleotides or genetic depletion, kills human and yeast dividing cells by inducing premature mitotic entry and apoptosis.

Thus, Pin1-catalyzed prolyl isomerization regulates the conformation and function of these phosphoprotein substrates and facilitates dephosphorylation because of the conformational specificity of some phosphatases. Thus, Pin1-dependent peptide bond isomerization is a critical post-phosphorylation regulatory mechanism, allowing cells to turn phosphoprotein function on or off with high efficiency and specificity during temporally regulated events, including the cell cycle (Lu et al., supra).

### SUMMARY OF THE INVENTION

A need exists for new diagnostic and therapeutic compounds for diseases characterized by uncontrolled cell proliferation and primarily malignancies associated with the Pin-b 1 subfamily of enzymes.

Accordingly, the invention is directed to modulators, e.g., inhibitors, of Pin1 and Pin1-related proteins and the use of such modulators for treatment of Pin1 associated states, e.g., for the treatment of cancer.

In one embodiment, the invention pertains, at least in part, to a method for treating a Pin1-associated state in a subject. The method includes administering to the subject an effective amount of a Pin1-modulating compound of formula (I):

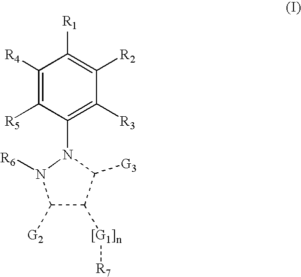

wherein

the dashed lines indicate a single or a double bond;

n is 0, 1, or 2;

G₁ is CH₂, CH, or N;

G₂ and G₃ are independently selected from the group consisting of O, OR₂, NR₂, NR₂R_{6,} hydrogen, alkyl, aryl;

R₁, R₂, R₃, R₄, R₅, R₆ and R₇ are each independently substituted or unsubstituted: alkyl, alkenyl, alkynyl, aryl, hydrogen, acyl, or any combination thereof;

such that the Pin1-associated state is treated.

In a second embodiment, the invention pertains, at least in part, to a method for treating cyclin D1 overexpression in a subject. This method includes administering to the subject an effective amount of a Pin1-modulating compound of formula (I):

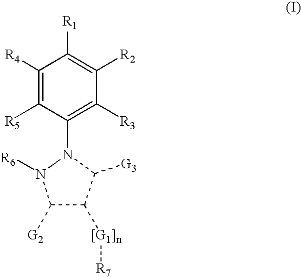

wherein

the dashed lines indicate a single or a double bond;

n is 0, 1, or 2;

G₁ is CH₂, CH, or N;

G₂ and G₃ are independently selected from the group consisting of O, OR₂, NR₂, NR₂R₆, hydrogen, alkyl, aryl;

R₁, R₂, R₃, R₄, R₅, R₆ and R₇ are each independently substituted or unsubstituted: alkyl, alkenyl, alkynyl, aryl, hydrogen, acyl, or any combination thereof;

such that the cyclin D1 overexpression is treated.

The invention also includes a packaged Pin1-associated state treatment. The packaged treatment comprises a Pin1-modulating compound of formula (I):

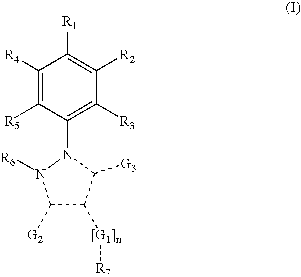

wherein

the dashed lines indicate a single or a double bond;

n is 0, 1, or 2;

G₁ is CH₂, CH, or N;

G₂ and G₃ are independently selected from the group consisting of O, OR₂, NR₂, NR₂R₆, hydrogen, alkyl, aryl;

R₁, R₂, R₃, R₄, R₅, R₆ and R₇ are each independently substituted or unsubstituted: alkyl, alkenyl, alkynyl, aryl, hydrogen, acyl, or any combination thereof;

packaged with instructions for using an effective amount of the Pin1-modulating compound to treat a Pin1 associated state.

The invention also includes a packaged cyclin D1 overexpression treatment. This packaged treatment include a Pin1-modulating compound of formula (I):

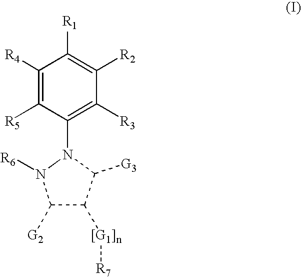

wherein

the dashed lines indicate a single or a double bond;

n is 0, 1, or 2;

G₁ is CH₂, CH, or N;

G₂ and G₃ are independently selected from the group consisting of O, OR₂, NR₂, NR₂R₆, hydrogen, alkyl, aryl;

R₁, R₂, R₃, R₄, R₅, R₆ and R₇ are each independently substituted or unsubstituted: alkyl, alkenyl, alkynyl, aryl, hydrogen, acyl, or any combination thereof;

packaged with instructions for using an effective amount of the Pin1-modulating compound to treat cyclin D1 overexpression.

In yet another embodiment, the invention also pertains, at least in part to a packaged cancer treatment, which includes a Pin1-modulating compound of formula (I):

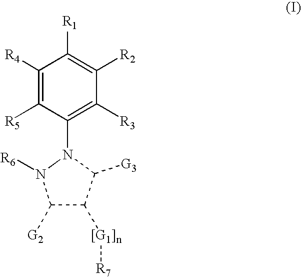

wherein

the dashed lines indicate a single or a double bond;

n is 0, 1, or 2;

G₁ is CH₂, CH, or N;

G₂ and G₃ are independently selected from the group consisting of O, OR₂, NR₂, NR₂R₆, hydrogen, alkyl, aryl;

R₁, R₂, R₃, R₄, R₅, R₆ and R₇ are each independently substituted or unsubstituted: alkyl, alkenyl, alkynyl, aryl, hydrogen, acyl, or any combination thereof;

packaged with instructions for using an effective amount of the Pin1-modulating compound to treat cancer.

In another embodiment, the invention pertains, at least in part, to a method for treating a Pin1-associated state in a subject. The method includes administering to a subject an effective amount of a combination of a Pin1-modulating compound of formula (I):

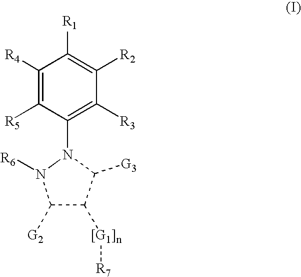

wherein

the dashed lines indicate a single or a double bond;

n is 0, 1, or 2;

G₁ is CH₂, CH, or N;

G₂ and G₃ are independently selected from the group consisting of O, OR₂, NR₂, NR₂R₆, hydrogen, alkyl, aryl;

R₁, R₂, R₃, R₄, R₅, R₆ and R₇ are each independently substituted or unsubstituted: alkyl, alkenyl, alkynyl, aryl, hydrogen, acyl, or any combination thereof; and

a hyperplastic inhibitory agent such that the Pin1 associated state is treated.

In another embodiment, the invention pertains, at least in part, to a method for treating cancer in a subject. The method includes administering to the subject an effective amount of a combination of a Pin1-modulating compound of formula (I):

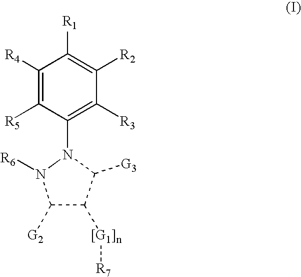

wherein

the dashed lines indicate a single or a double bond;

n is 0, 1, or 2;

G₁ is CH₂, CH, or N;

G₂ and G₃ are independently selected from the group consisting of O, OR₂, NR₂, NR₂R₆, hydrogen, alkyl, aryl;

R₁, R₂, R₃, R₄, R₅, R₆ and R₇ are each independently substituted or unsubstituted: alkyl, alkenyl, alkynyl, aryl, hydrogen, acyl, or any combination thereof; and

a hyperplastic inhibitory agent such that the cancer is treated.

In an additional embodiment, the invention is a method for treating cyclin D1 overexpression in a subject. The method includes administering to the subject an effective amount of a combination of a Pin1-modulating compound of formula (I):

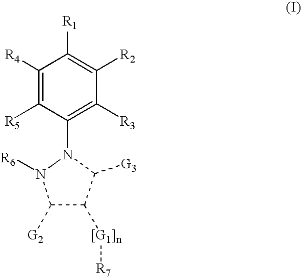

wherein

the dashed lines indicate a single or a double bond;

n is 0, 1, or 2;

G₁ is CH₂, CH, or N;

G₂ and G₃ are independently selected from the group consisting of O, OR₂, NR₂, NR₂R₆, hydrogen, alkyl, aryl;

R₁, R₂, R₃, R₄, R₅, R₆ and R₇ are each independently substituted or unsubstituted: alkyl, alkenyl, alkynyl, aryl, hydrogen, acyl, or any combination thereof; and

a hyperplastic inhibitory agent such that the cyclin D1 overexpression is treated.

Another embodiment of the invention is a Pin1-modulator comprising formula (I):

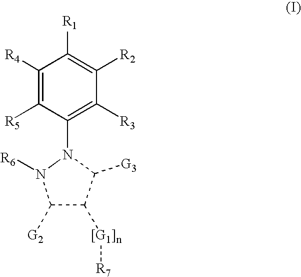

wherein

the dashed lines indicate a single or a double bond;

n is 0, 1, or 2;

G₁ is CH₂, CH, or N;

G₂ and G₃ are independently selected from the group consisting of O, OR₂, NR₂, NR₂R₆, hydrogen, alkyl, aryl; and

R₁, R₂, R₃, R₄, R₅, R₆ and R₇ are each independently substituted or unsubstituted: alkyl, alkenyl, alkynyl, aryl, hydrogen, acyl, or any combination thereof.

Another embodiment of the invention is a pharmaceutical composition comprising a Pin1-modulating compound as prepared according to the methodology of this invention, and a pharmaceutically acceptable carrier.

### DETAILED DESCRIPTION OF THE INVENTION

The invention is directed to modulators, e.g., inhibitors, of Pin1 and Pin1-related proteins and the use of such modulators for treatment of Pin1 associated states, e.g., for the treatment of cancer.

In one embodiment, the invention pertains, at least in part, to a method for treating a Pin1-associated state in a subject. The method includes administering to the subject an effective amount of a Pin1-modulating compound of formula (I):

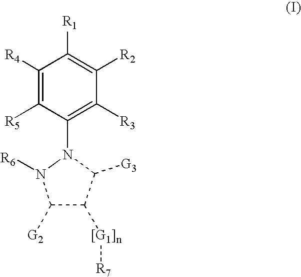

wherein

the dashed lines indicate a single or a double bond;

n is 0, 1, or 2;

G₁ is CH₂, CH, or N;

G₂ and G₃ are independently selected from the group consisting of O, OR₂, NR₂, NR₂R₆, hydrogen, alkyl, aryl;

R₁, R₂, R₃, R₄, R₅, R₆ and R₇ are each independently substituted or unsubstituted: alkyl, alkenyl, alkynyl, aryl, hydrogen, acyl, or any combination thereof;

such that the Pin1-associated state is treated.

The term “Pin1-associated state” or “Pin1 associated disorder” includes disorders and states (e.g., a disease state) which are associated with abnormal cell growth, abnormal cell proliferation, or aberrant levels of Pin1 (e.g., Pin1 protein or nucleic acid). Pin1-associated states include states resulting from an elevation in the expression of cyclin D1 and/or Pin1. Pin1-associated states also include states resulting from an elevation in the phosphorylation level of c-Jun, particularly phosphorylation of c-Jun on Ser^{63/73}-Pro and/or from an elevation in the level of c-Jun amino terminal kinases (JNKs) present in a cell. Pin1-associated states include neoplasia, cancer, undesirable cell growth, and/or tumor growth. Pin1-associated states include states caused by DNA damage, an oncogenic protein (i.e. Ha-Ras), loss of or reduced expression of a tumor suppressor (i.e. Brcal), and/or growth factors.

Pin1 is an important regulator of cyclin D1 expression. Due to Pin1's role in regulating the expression of cyclin D1, many of the tumor causing effects of cyclin D1 can be regulated through Pin1. In particular, inhibitors of Pin1 can also be used to treat, inhibit, and/or prevent undesirable cell growth, e.g., tumors, neoplasia, and/or cancer associated with aberrant cyclin D1 expression in a subject.

Other examples of Pin1 associated states include, but are not limited to, for example, those tumor types disclosed in Table 4.

The term “treated,” “treating” or “treatment” includes the diminishment or alleviation of at least one symptom associated or caused by the state, disorder or disease being treated. In certain embodiments, the treatment comprises the induction of a Pin1 inhibited state, followed by the activation of the Pin1 modulating compound, which would in turn diminish or alleviate at least one symptom associated or caused by the Pin1 associated state, disorder or disease being treated. For example, treatment can be diminishment of one or several symptoms of a disorder or complete eradication of a disorder.

The term “subject” is intended to include organisms, e.g., prokaryotes and eukaryotes, which are capable of suffering from or afflicted with a Pin1 associated disorder. Examples of subjects include mammals, e.g., humans, dogs, cows, horses, pigs, sheep, goats, cats, mice, rabbits, rats, and transgenic non-human animals. In certain embodiments, the subject is a human, e.g., a human suffering from, at risk of suffering from, or potentially capable of suffering from a Pin1 associated disorder.

The language “Pin1 modulating compound” refers to compounds that modulate, e.g., inhibit, promote, or otherwise alter, the activity of Pin1. Pin1 modulating compounds include both Pin1 agonists and embodiments, the Pin1 modulating compound induces a Pin1 inhibited-state. Examples of Pin1 modulating compounds include compounds of formula (I). Additional examples of Pin1 modulating compounds include compounds of Table 1 or Table 2, or derivatives thereof. In certain embodiments, the Pin1 modulating compounds include compounds that interact with the PPI or the WW domain of Pin1. In certain embodiments, the Pin1 modulating compound is substantially specific to Pin1. The phrase “substantially specific for Pin1” is intended to include inhibitors of the invention that have a Kᵢ or K_{d} that is at least 2, 3, 4, 5, 10, 15, or 20 times less than the Kᵢ or K_{d} for other peptidyl prolyl isomerases, e.g., hCyP-A, hCyP-B, hCyP-C, NKCA, hFKBP-12, hFKBP-13, and hFKBP-25.

The language “Pin1 inhibiting compound” includes compounds that reduce or inhibit the activity of Pin1. Examples of Pin1 inhibiting compounds include compounds of formula (I). Additional examples of Pin1 inhibiting compounds include compounds of Table 1 or Table 2, or derivatives thereof. In certain embodiments, the Pin1 inhibiting compounds include compounds that interact with the PPI or the WW domain of Pin1.

In certain embodiments the inhibitors have a Kᵢ for Pin1 of less than 0.2 mM, less than 0.1 mM, less than 750 μM, less than 500 μM, less than 250 μM, less than 100 μM, less than 50 μM, less than 500 nM, less than 250 nM, less than 50 nM, less than 10 nM, less than 5 nM, or or less than 2 nM.

The language “Pin1 inhibited-state” is intended to include states in which one activity of Pin1 is inhibited in cells, e.g., cells in a subject, that have been treated with a Pin1 modulating compound. “Pin1 inbited-state is also intended to include states wherein the Pin1 modulating compound is administered to a subject, allowed to remain in a preactivated state, and subsequently activated by a stimulus. The stimulus may be selected from a natural event, artificial event, or the combination thereof. For example, the natural event may be the action of an enzyme and/or the artificial event may be the addition of a hyperplastic inhibitory agent or the addition of energy to the subjects system in any manner that achieves activation, e.g., by radiation, e.g., by light with a wavelength greater than about 400 nm, e.g., greater than about 600 nm, e.g., greater than about 620 nm, e.g., greater than about 630 nm, e.g., greater than about 640 nm, e.g., greater than about 650 nm. In one embodiment, the cells enter a Pin1 inhibited-state for a designated period of time prior to activation of the modulating compound sufficient to allow the modulation the activity of Pin1 by the activated modulating compound. In certain embodiments of the invention, the designated period of time prior to activation is greater than about 1 hour, e.g., greater than about 2 hours, e.g., greater than about 3 hours, e.g., greater than about 6 hours, e.g., greater than about 12 hours, e.g., greater than about 24 hours, e.g., greater than about 36 hours, e.g., greater than about 48 hours, e.g., greater than about 72 hours. In a specific embodiment, the designated period of time prior to activation is 3 days. In one embodiment, the Pin1 modulating compound is preactivated prior to administration to a subject followed by the introduction of at least one stimulus sufficient to allow the modulation the activity of Pin1 by the modulating compound. In certain embodiment of the invention, the activity of the modulating compound is enhanced by the entrance of the cells, e.g., cells of a subject, into a Pin1 inhibited state.

In one embodiment of the invention, the Pin1 modulating compounds of the invention have a characteristic inhibition profile (CIP) and have an effective cytotoxicity, e.g., effective to treat a Pin1 associated state. The Pin1-modulating compounds described herein may be substituted with any substituent that allows the Pin1-modulating compound to perform its intended function. In certain embodiments the Pin1-modulating compounds described herein may be substituted with any substituent which allows the Pin1-modulating compound to perform its intended function, possess a CIP, and/or be effectively cytotoxic, as defined herein. The cytotoxicity of the compounds can be determined by using the CPCA given in Example 1. The measurement of the activity of the Pin1-modulating compounds in the determination the inhibition constant at 50% inhibition of enzyme activity (IC₅₀), which is used to characterize the CIP, may be performed by using the analysis described in Example 2. An ordinarily skilled artisan would be able to use data generated by the assays to modify substituents on the Pin1 modulating compounds to obtain effectively cytotoxic Pin1 modulating compounds with characteristic inhibition profiles.

The term “characteristic inhibition profile (CIP)” is a characterization of the modulating compound of the invention such that the Pin1-associated state is inhibited. Characterization of the modulating compounds includes measurement of the inhibition constant at 50% inhibition of enzyme activity (IC₅₀). Compounds that demonstrate a CIP include modulating compounds with and IC₅₀ of less than about 40 μM. In certain embodiments of the invention, the IC₅₀ is between about 10-40 μM. In additional embodiments, the IC₅₀ is between about 1-10 μM. In certain embodiments, the IC₅₀ is less than about 1 μM.

The term “effective cytotoxicity” or “effectively cytotoxic” includes cytotoxicities of Pin1-modulating compounds which allow the Pin1-modulating compound to perform its intended function, e.g., treat Pin1 associated states. Cytotoxicities can be measured, for example, by using the Cell Based Cytotoxicity Assay (CBCA) method described in Example 1. In one embodiment, the Pin1-modulating compound has a cytotoxicity (as measured by the CBCA in Example 1) of 50 μM or less, 45 μM or less, 40 μM or less, 35 μM or less, 30 μM or less, 25 μM or less, 20 μM or less, 15 μM or less, 10 μM or less, 9 μM or less, 8 μM or less, 7 μM or less, 6 μM or less, 5 μM or less, 4 μM or less, 3 μM or less, 2 μM or less, 1 μM or less, 0.9 μM or less, 0.8 μM or less, 0.7 μM or less, 0.6 μM or less, 0.5 μM or less, 0.4 μM or less, or, preferably, 0.3 μM or less, or 0.05 μM or less. Values and ranges included and/or intermediate of the values set forth herein are also intended to be within the scope of the present invention.

In one embodiment, the Pin1 modulating compounds of the invention are substantially soluble, e.g., water soluble, and have an effective cytotoxicity, e.g., effective to treat a Pin1 associated state. Methods for altering the solubility of organic compounds are known in the art. For example, one of ordinary skill in the art will be able to modify the Pin1 modulating compounds of the invention such that they have a desirable logP. Ordinarily skilled artisans will be able to modify the compounds by adding and removing hydrophilic and hydrophobic moieties, such that a Pin1-modulating compound with a desired solubility is obtained. The Pin1-modulating compounds described herein may be substituted with any substituent which allows the Pin1-modulating compound to perform its intended function, be substantially soluble, and/or be effectively cytotoxic, as defined herein. For example, an ordinarily skilled artisan would understand that the addition of heteroatoms (hydroxy, amino, nitro, carboxylic acid groups, etc.) or other polar moieties would generally increase the solubility of the Pin1 modulating compound in water, while addition of non-polar moieties such as aryl or alkyl groups would generally decrease the solubility of the compound in water. The Pin1 modulating compound can then be tested for substantial solubility by determining the logP value (e.g., by using a log octanol-water partition coefficient program such as “KOWWIN” (Meylan, W. M. and P. H. Howard. 1995. Atom/fragment contribution method for estimating octanol-water partition coefficients. J Pharm. Sci. 84: 83-92, incorporated herein by reference in its entirety). An ordinarily skilled artisan would be able to use data generated by these programs and assays to modify substituents on the Pin1 modulating compounds to obtain substantially soluble and effectively cytotoxic Pin1 modulating compounds.

The term “substantially soluble” includes solubilities (e.g., aqueous solubilities) of Pin1-modulating compounds that allow the Pin1-modulating compounds to perform their intended function, e.g., treat Pin1 associated states. The solubility of a particular Pin1-modulating compound can be measured by any method known in the art, e.g., experimentally, computationally, etc. For example, one method for determining the solubility of a compound computationally is by calculating logP values using a log octanol-water partition coefficient program (KOWWIN). In one embodiment, the Pin1-modulating compounds of the invention have logP values less than Pin1-modulating, e.g., less than 6.6. In a further embodiment, the Pin1-modulating compounds of the invention may have a logP value between about 1 to about 6, between about 1 to about 5, between about 1.5 to about 5, between about 2 to about 5, between about 2.5 to about 4.5, between about 2.75 to about 4.25, between about 3.0 to about 4.0, between about 3.25 to about 4.0, between about 3.5 to about 4.0, and between about 3.5 to about 3.75. Values and ranges included and/or intermediate of the values set forth herein are also intended to be within the scope of the present invention. In another embodiment, the aqueous solubility of the compound is about 0.01 mg/L or greater, about 0.1 mg/L or greater, about 1 mg/L or greater, or about 2 mg/L or greater.

In certain embodiments of the invention, G₂ is O, hydrogen, CH₃, thiophene, or nitrophenyl; G₃ is O, H, or OH; and R₆ is H or CH₃. In addition, in certain embodiments of the invention, R₇ is a phenyl, a pyridine, an indole, an indene, a pyrazole, a benzoimidazole, a thiophene, a naphthalene, a morpholine, a pyrrolidine, a piperidine, a furan, a tetrahydrofuran, a benzo[1,3]dioxole, a pyrole, a cyclohexene, a furazan-2-oxide, a thioxo-thiazolidinone, an isobenzofuranone, a benzo[4,5]imidazo[1,2-a]pyridine, an isobutyl, a derivative thereof, or a combination thereof, wherein the combination may further comprise alkyl, alkenyl, alkynyl, or acyl groups.

Additionally, in particular embodiments of the invention, R₇ is substituted with substituents selected from the group consisting of H, O, OH, Cl, Br, F, I, OEt, OMe, CO₂H, propenyloxy, acetyl, isopropyl, butyloxy, benzyloxy, propyloxy, morpholino, dimethylamino, NO₂, sulfonamide, CO₂CH₂CH₃, —OCH₂CO₂CH₂CH₃, benzene sulfonate, acetamide, Me, t-butyl, propargyl, naphthyl, naphthyloxy, propargyloxy, hexyloxy, octyloxy, dipropylamino, ethylmethylamino, propyloxy, iso-propyl, benzyl, phenyl, methylsulfanyl, phenylsulfanyl, naphthylsulfanyl, benzoyl, , CO₂CH₃, —OCH₂CO₂CH₃, —OCH₂CO₂H, —OCH₂CH₂O—R₃, —CH₂CH₂O—R₃, —OCH₂CH₂S—R₃, derivatives thereof, and combinations thereof.

In particular embodiments of formula (I), R₁, R₂, R₃, R₄, and R₅ are each independently selected from the group consisting of CH₃, Br, I, Cl, H, F, CO₂CH₂CH₃, CF₃, Et, combinations thereof, and derivatives thereof.

The term “derivative” is intended to include isomers, modification, e.g., addition or removal, of substituents on the Pin1-modulating compound, and pharmaceutically acceptable salts thereof, as well as formulation, such that the Pin1-modulating compound treats the Pin1-associated state.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In an embodiment, a straight chain or branched chain alkyl has 10 or fewer carbon atoms in its backbone (e.g., C₁-C₁₀for straight chain, C₃-C₁₀ for branched chain), and more preferably 6 or fewer. Likewise, preferred cycloalkyls have from 4-7 carbon atoms in their ring structure, and more preferably have 5 or 6 carbons in the ring structure.

Moreover, the term alkyl includes both “unsubstituted alkyls” and “substituted alkyls”, the latter of which refers to alkyl moieties having substituents replacing a hydrogen on one or more carbons of the hydrocarbon backbone. Such substituents can include, for example, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “aralkyl” moiety is an alkyl substituted with an aryl (e.g., phenylmethyl (benzyl)). The term “alkyl” also includes the side chains of natural and unnatural amino acids. Examples of halogenated alkyl groups include fluoromethyl, difluoromethyl, trifluoromethyl, chloromethyl, dichloromethyl, trichloromethyl, perfluoromethyl, perchloromethyl, perfluoroethyl, perchloroethyl, etc.

The term “aryl” includes groups, including 5- and 6-membered single-ring aromatic groups that may include from zero to four heteroatoms, for example, benzene, phenyl, pyrrole, furan, thiophene, thiazole, isothiaozole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles”, “heterocycles,” “heteroaryls” or “heteroaromatics”. The aromatic ring can be substituted at one or more ring positions with such substituents as described above, as for example, halogen, hydroxyl, alkoxy, alkylcarbonyloxy, arylcarbonyloxy, alkoxycarbonyloxy, aryloxycarbonyloxy, carboxylate, alkylcarbonyl, alkylaminoacarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “alkenyl” includes unsaturated aliphatic groups analogous in length and possible substitution to the alkyls described above, but which contain at least one double bond.

For example, the term “alkenyl” includes straight-chain alkenyl groups (e.g., ethenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups that include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls”, the latter of which refers to alkenyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulthydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups that include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term alkynyl includes both “unsubstituted alkynyls” and “substituted alkynyls”, the latter of which refers to alkynyl moieties having substituents replacing a hydrogen on one or more carbons of the hydrocarbon backbone. Such substituents can include, for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, “lower alkyl” as used herein means an alkyl group, as defined above, but having from one to five carbon atoms in its backbone structure. “Lower alkenyl” and “lower alkynyl” have chain lengths of, for example, 2-5 carbon atoms.

The term “acyl” includes compounds and moieties which contain the acyl radical (CH₃CO—) or a carbonyl group. The term “substituted acyl” includes acyl groups where one or more of the hydrogen atoms are replaced by for example, alkyl groups, alkynyl groups, halogens, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. The term “acylamino” includes moieties wherein an acyl moiety is bonded to an amino group. For example, the term includes alkylcarbonylamino, arylcarbonylamino, carbamoyl and ureido groups.

The term “aroyl” includes compounds and moieties with an aryl or heteroaromatic moiety bound to a carbonyl group. Examples of aroyl groups include phenylcarboxy, naphthyl carboxy, etc.

The terms “alkoxyalkyl”, “alkylaminoalkyl” and “thioalkoxyalkyl” include alkyl groups, as described above, which further include oxygen, nitrogen or sulfur atoms replacing one or more carbons of the hydrocarbon backbone, e.g., oxygen, nitrogen or sulfur atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups and may include cyclic groups such as cyclopentoxy. Examples of substituted alkoxy groups include halogenated alkoxy groups. The alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulthydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “amine” or “amino” includes compounds where a nitrogen atom is covalently bonded to at least one carbon or heteroatom. The term “alkyl amino” includes groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group that is bound to at least one alkyl group and at least one aryl group. The term “alkaminoalkyl” refers to an alkyl, alkenyl, or alkynyl group bound to a nitrogen atom that is also bound to an alkyl group.

The term “amide” or “aminocarboxy” includes compounds or moieties that contain a nitrogen atom that is bound to the carbon of a carbonyl or a thiocarbonyl group. The term includes “alkaminocarboxy” groups that include alkyl, alkenyl, or alkynyl groups bound to an amino group bound to a carboxy group. It includes arylaminocarboxy groups that include aryl or heteroaryl moieties bound to an amino group which is bound to the carbon of a carbonyl or thiocarbonyl group. The terms “alkylaminocarboxy,” “alkenylaminocarboxy,” “alkynylaminocarboxy,” and “arylaminocarboxy” include moieties wherein alkyl, alkenyl, alkynyl and aryl moieties, respectively, are bound to a nitrogen atom which is in turn bound to the carbon of a carbonyl group.

The term “carbonyl” or “carboxy” includes compounds and moieties which contain a carbon connected with a double bond to an oxygen atom, and tautomeric forms thereof. Examples of moieties that contain a carbonyl include aldehydes, ketones, carboxylic acids, amides, esters, anhydrides, etc. The term “carboxy moiety” or “carbonyl moiety” refers to groups such as “alkylcarbonyl” groups wherein an alkyl group is covalently bound to a carbonyl group, “alkenylcarbonyl” groups wherein an alkenyl group is covale group, “alkynylcarbonyl” groups wherein an alkynyl group is covalently bound to a carbonyl group, “arylcarbonyl” groups wherein an aryl group is covalently attached to the carbonyl group. Furthermore, the term also refers to groups wherein one or more heteroatoms are covalently bonded to the carbonyl moiety. For example, the term includes moieties such as, for example, aminocarbonyl moieties, (wherein a nitrogen atom is bound to the carbon of the carbonyl group, e.g., an amide), aminocarbonyloxy moieties, wherein an oxygen and a nitrogen atom are both bond to the carbon of the carbonyl group (e.g., also referred to as a “carbamate”). Furthermore, aminocarbonylamino groups (e.g., ureas) are also include as well as other combinations of carbonyl groups bound to heteroatoms (e.g., nitrogen, oxygen, sulfur, etc. as well as carbon atoms). Furthermore, the heteroatom can be further substituted with one or more alkyl, alkenyl, alkynyl, aryl, aralkyl, acyl, etc. moieties.

The term “thiocarbonyl” or “thiocarboxy” includes compounds and moieties which contain a carbon connected with a double bond to a sulfur atom. The term “thiocarbonyl moiety” includes moieties that are analogous to carbonyl moieties. For example, “thiocarbonyl” moieties include aminothiocarbonyl, wherein an amino group is bound to the carbon atom of the thiocarbonyl group, furthermore other thiocarbonyl moieties include, oxythiocarbonyls (oxygen bound to the carbon atom), aminothiocarbonylamino groups, etc.

The term “ether” includes compounds or moieties that contain an oxygen bonded to two different carbon atoms or heteroatoms. For example, the term includes “alkoxyalkyl” which refers to an alkyl, alkenyl, or alkynyl group covalently bonded to an oxygen atom which is covalently bonded to another alkyl group.

The term “ester” includes compounds and moieties that contain a carbon or a heteroatom bound to an oxygen atom that is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above.

The term “thioether” includes compounds and moieties which contain a sulfur atom bonded to two different carbon or hetero atoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom that is bonded to an alkyl group. Similarly, the term “alkthioalkenyls” and alkthioalkynyls” refer to compounds or moieties wherein an alkyl, alkenyl, or alkynyl group is bonded to a sulfur atom that is covalently bonded to an alkynyl group.

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

The terms “polycyclyl” or “polycyclic radical” include moieties with two or more rings (e.g., cycloalkyls, cycloalkenyls, cycloalkynyls, aryls and/or heterocyclyls) in which two or more carbons are common to two adjoining rings, e.g., the rings are “fused rings”. Rings that are joined through non-adjacent atoms are termed “bridged” rings. Each of the rings of the polycycle can be substituted with such substituents as described above, as for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, aralkylaminocarbonyl, alkenylaminocarbonyl, alkylcarbonyl, arylcarbonyl, aralkylcarbonyl, alkenylcarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur and phosphorus.

The term “heterocycle” or “heterocyclic” includes saturated, unsaturated, aromatic (“heteroaryls” or “heteroaromatic”) and polycyclic rings which contain one or more heteroatoms. Examples of heterocycles include, for example, benzodioxazole, benzofuran, benzoimidazole, benzothiazole, benzothiophene, benzoxazole, deazapurine, furan, indole, indolizine, imidazole, isooxazole, isoquinoline, isothiaozole, methylenedioxyphenyl, napthridine, oxazole, purine, pyrazine, pyrazole, pyridazine, pyridine, pyrimidine, pyrrole, quinoline, tetrazole, thiazole, thiophene, and triazole. Other heterocycles include morpholine, piprazine, piperidine, thiomorpholine, and thioazolidine. The heterocycles may be substituted or unsubstituted. Examples of substituents include, for example, halogen, hydroxyl, alkylcarbonyloxy, arylcarbonyloxy, alkoxycarbonyloxy, aryloxycarbonyloxy, carboxylate, alkylcarbonyl, alkoxycarbonyl, alkylaminoacarbonyl, aralkylaminocarbonyl, alkenylaminocarbonyl, alkylcarbonyl, arylcarbonyl, aralkylcarbonyl, alkenylcarbonyl, aminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

It will be noted that the structures of some of the compounds of this invention include asymmetric carbon atoms. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis. Furthermore, the structures and other compounds and moieties discussed in this application also include all tautomers thereof. Compounds described herein may be obtained though art recognized synthesis strategies.

TABLE 1

| | | |
|---|---|---|
| 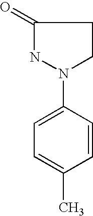 | 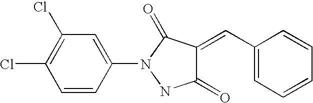 | 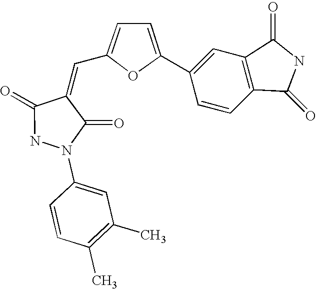 |
| 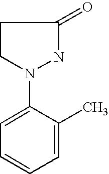 | 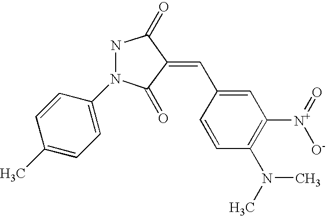 | 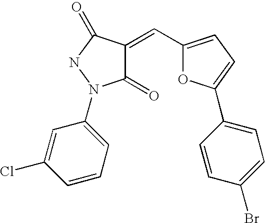 |
| 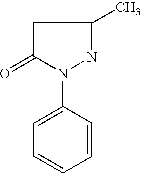 | 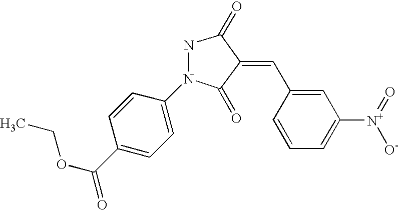 | 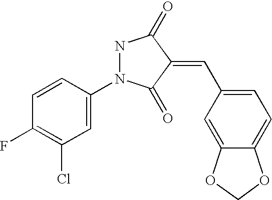 |
| 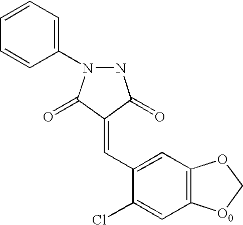 | 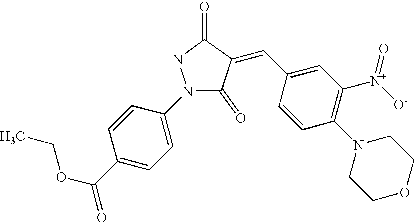 | 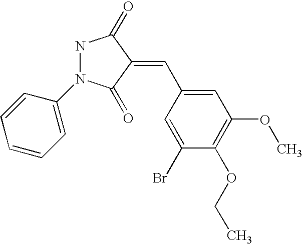 |
| 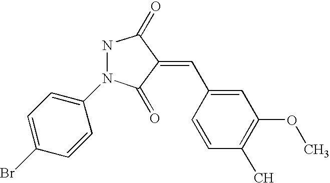 | 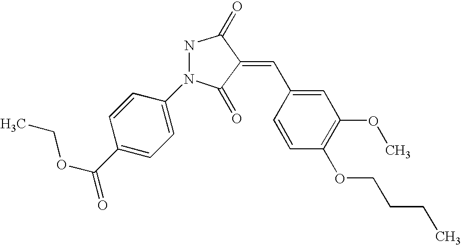 | 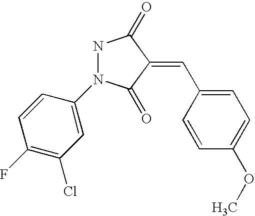 |
| 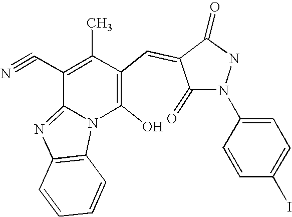 | 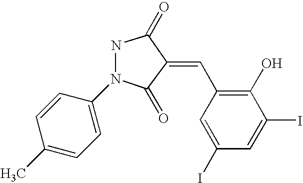 | 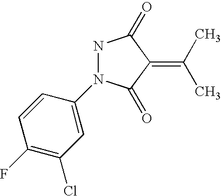 |
| 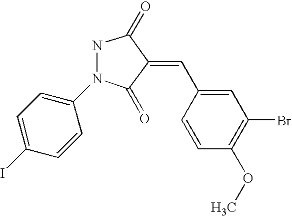 | 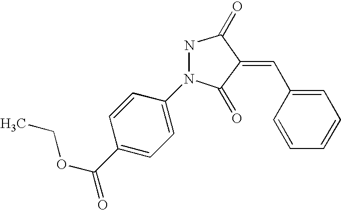 | 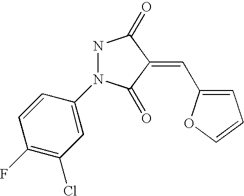 |
| 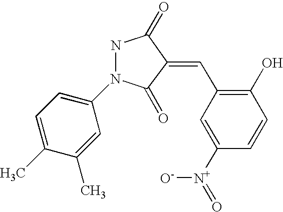 | 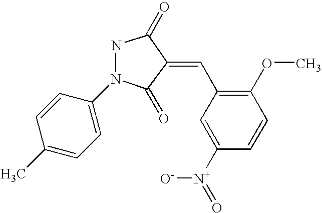 | 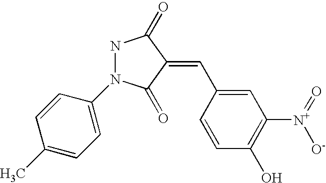 |
| 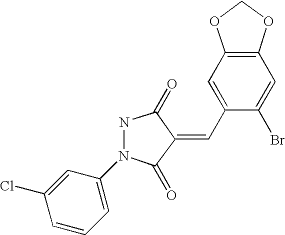 | 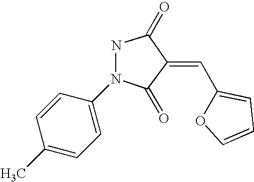 | 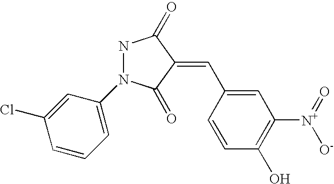 |
| 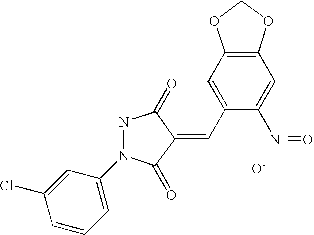 | 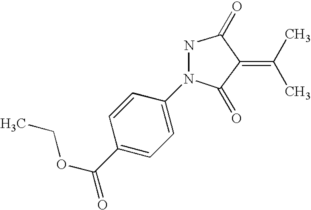 | 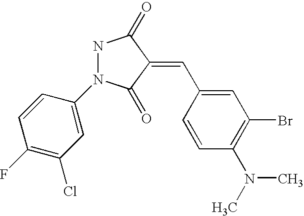 |
| 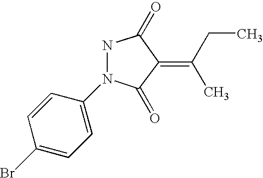 | 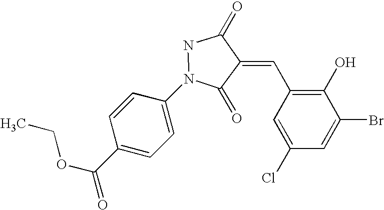 | 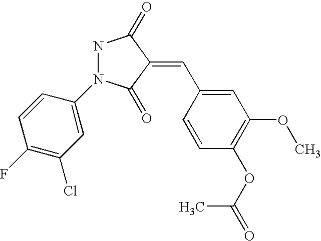 |
| 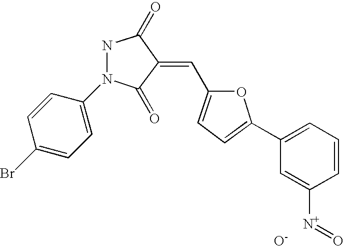 | 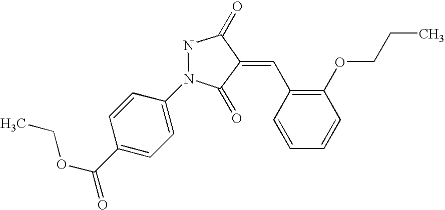 | 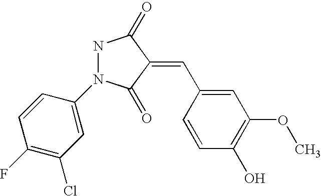 |
| 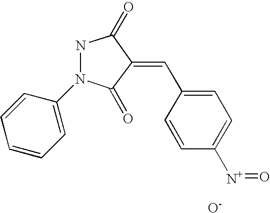 | 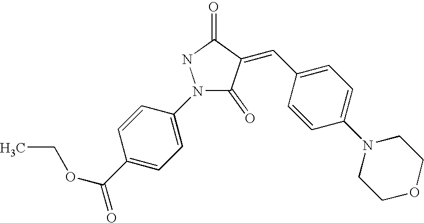 | 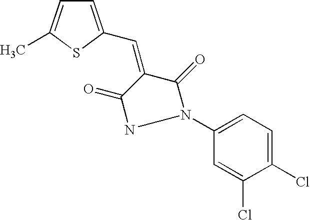 |
| 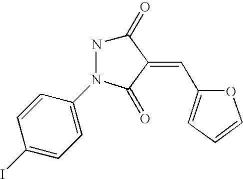 | 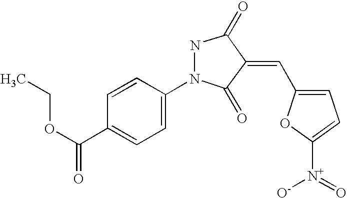 | 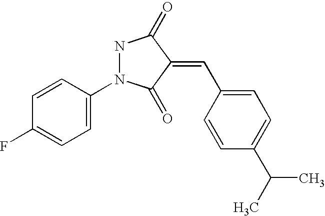 |
| 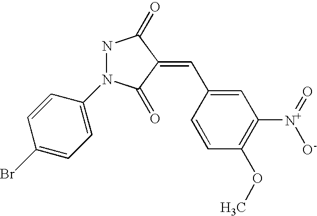 | 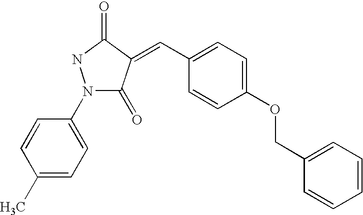 | 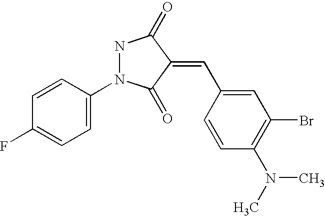 |
| 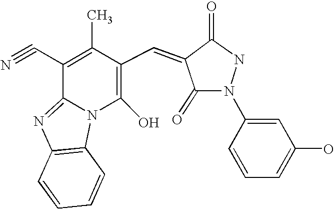 | 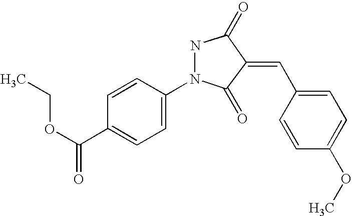 | 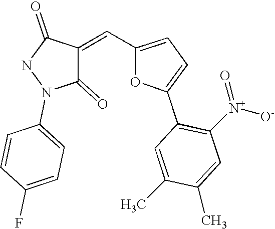 |
| 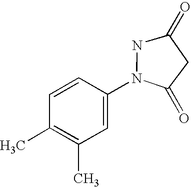 | 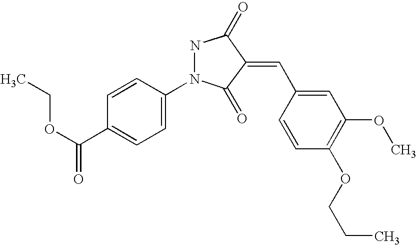 | 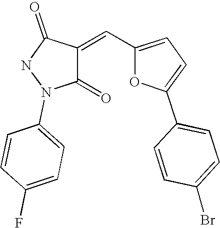 |
| 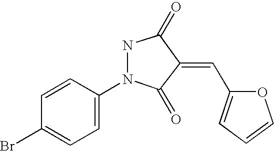 | 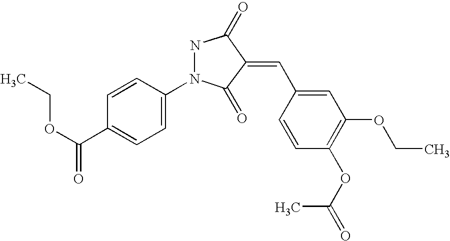 | 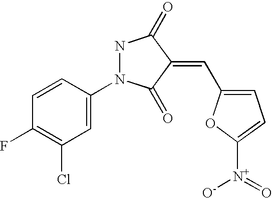 |
| 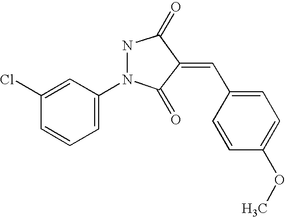 | 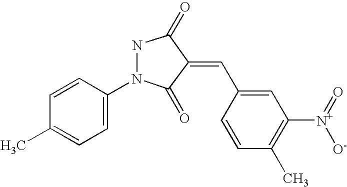 | 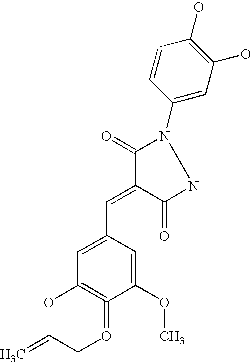 |
| 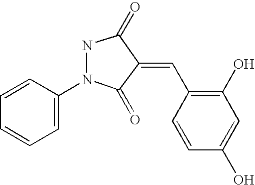 | 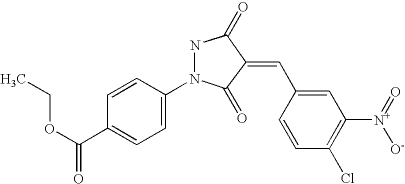 | 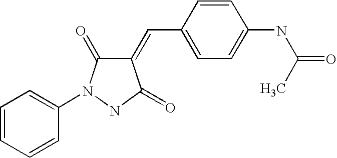 |
| 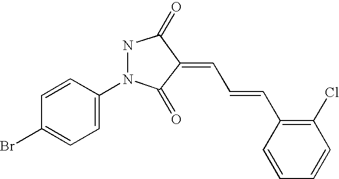 | 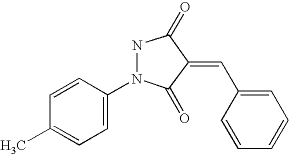 | 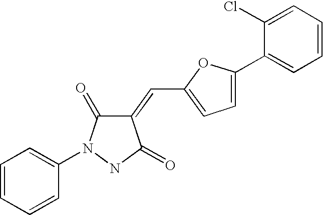 |
| 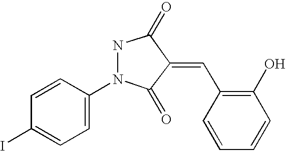 | 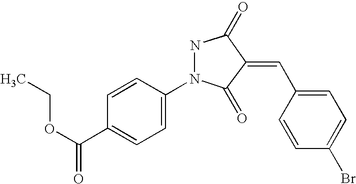 | 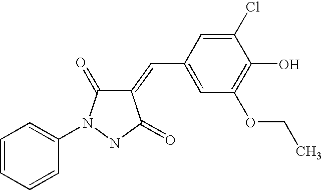 |
| 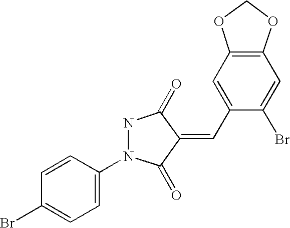 | 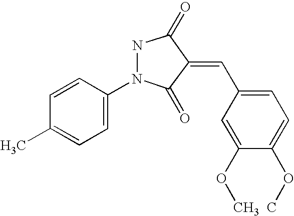 | 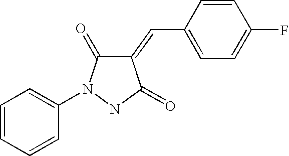 |
| 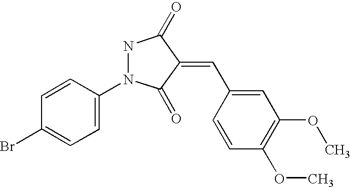 | 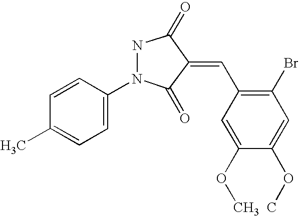 | 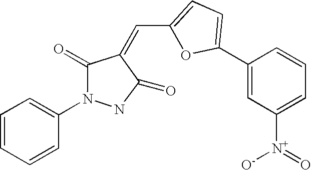 |
| 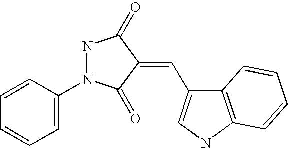 | 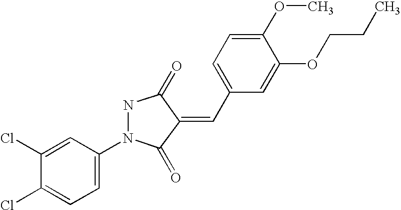 | 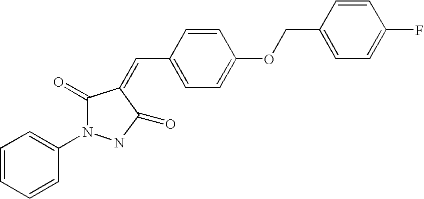 |
| 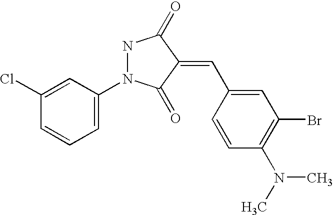 | 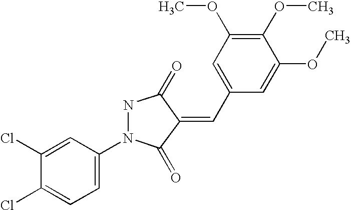 | 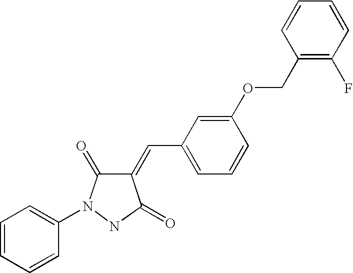 |
| 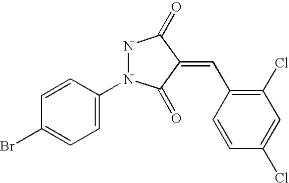 | 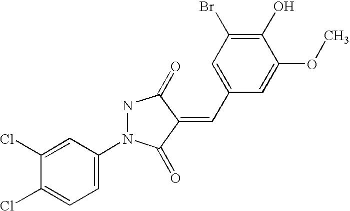 | 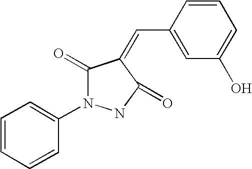 |
| 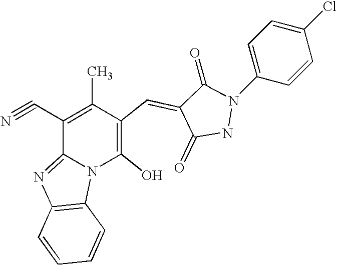 | 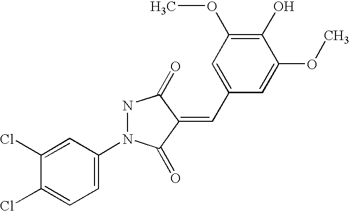 | 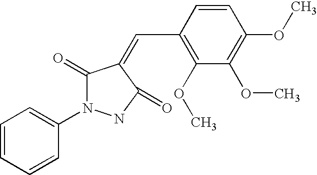 |
| 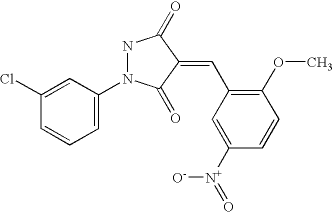 | 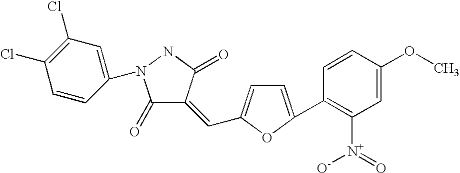 | 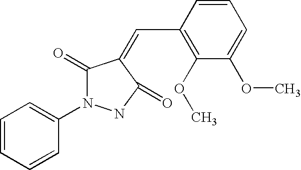 |
| 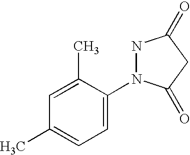 | 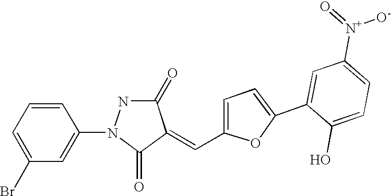 | 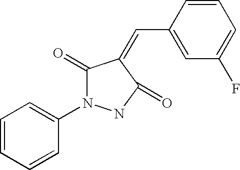 |
| 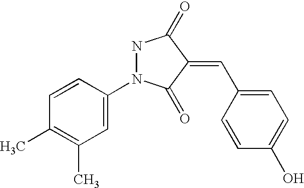 | 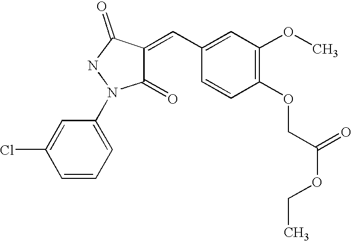 | 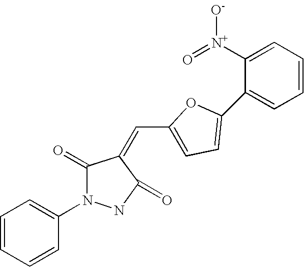 |
| 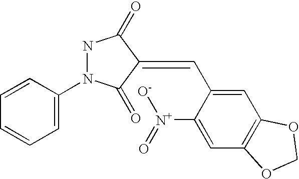 | 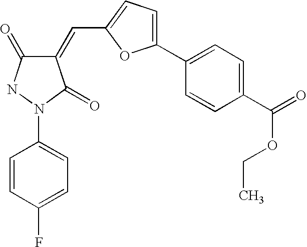 | 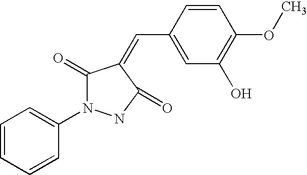 |
| 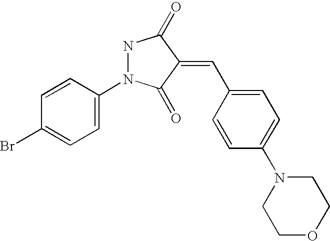 | 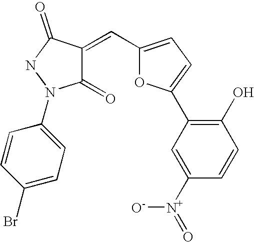 | 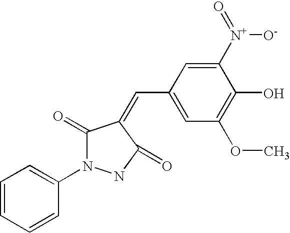 |
| 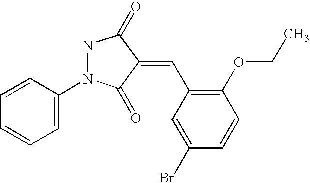 | 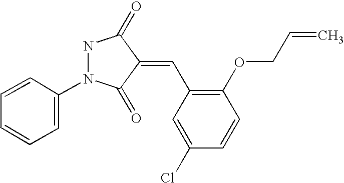 | 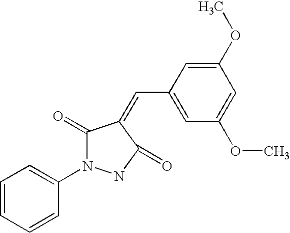 |
| 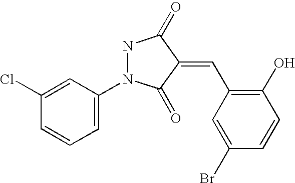 | 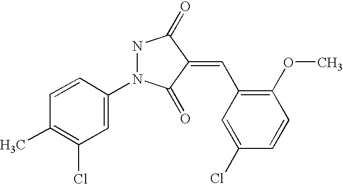 | 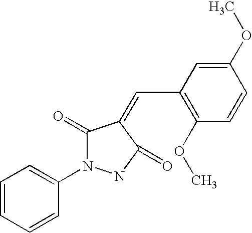 |
| 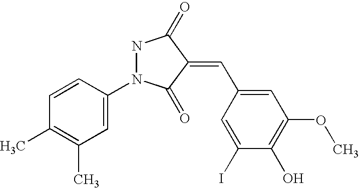 | 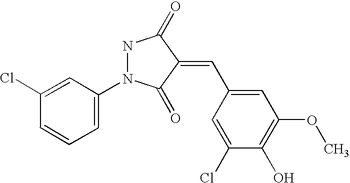 | 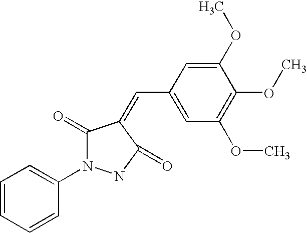 |
| 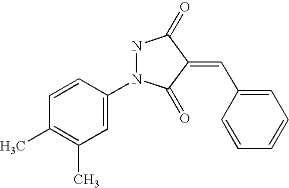 | 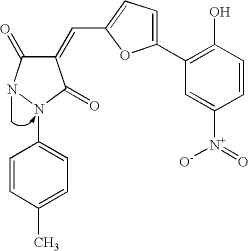 | 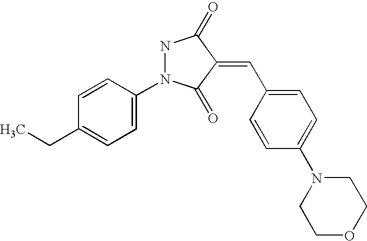 |
| 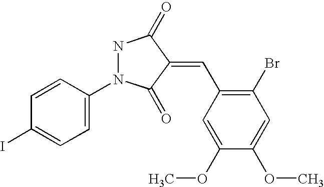 | 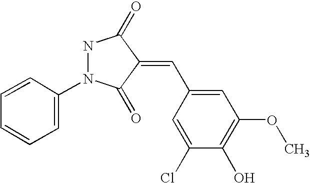 | 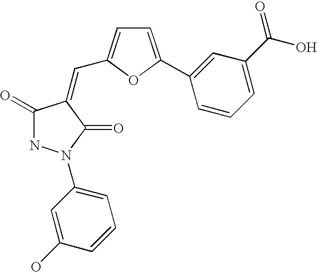 |
| 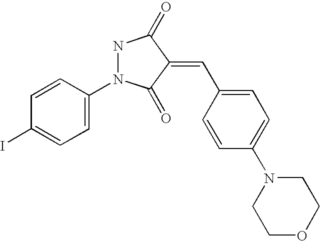 | 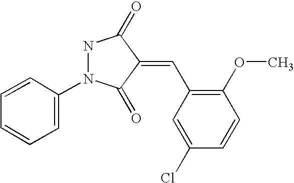 | 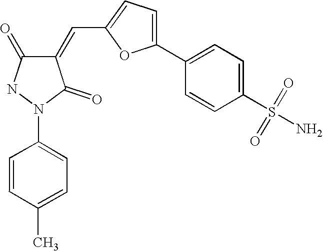 |
| 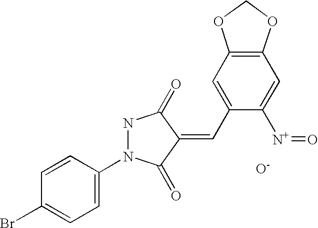 | 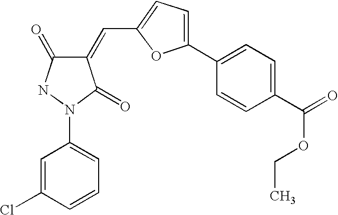 | 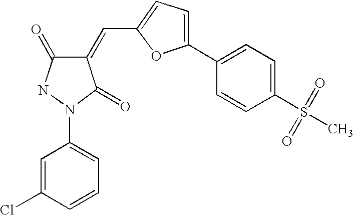 |
| 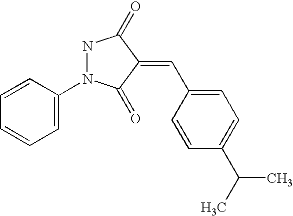 | 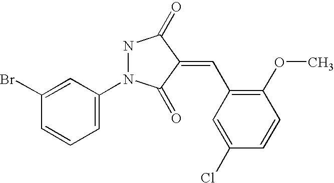 | 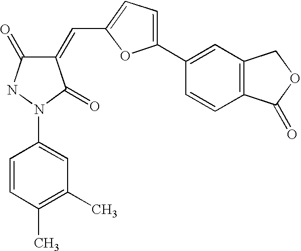 |
| 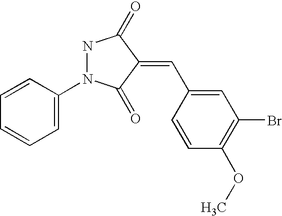 | 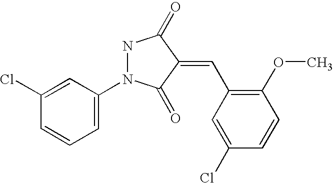 | 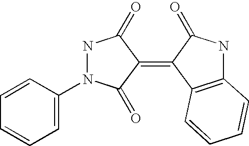 |
| 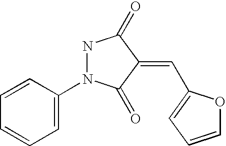 | 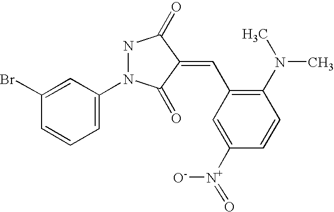 | 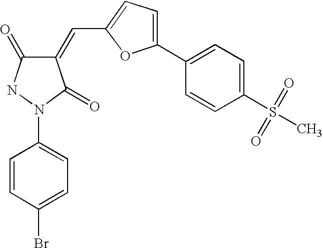 |
| 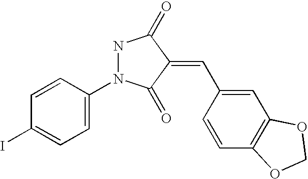 | 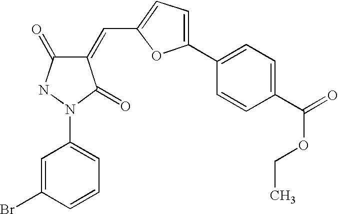 | 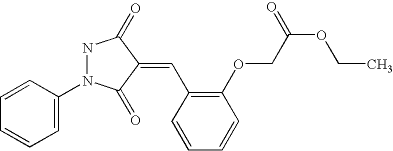 |
| 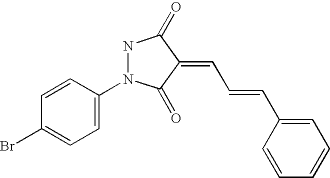 | 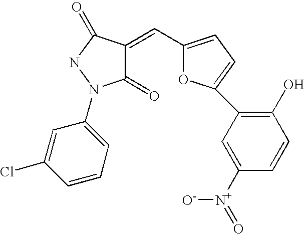 | 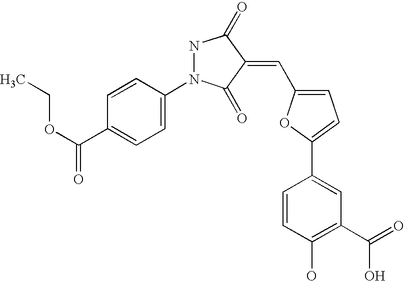 |
| 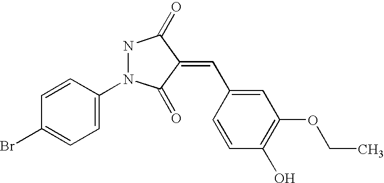 | 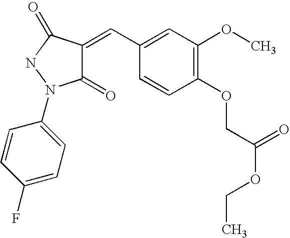 | 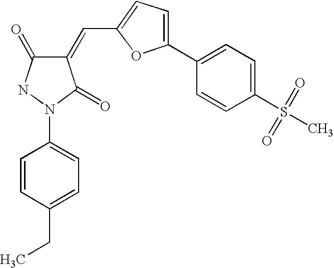 |
| 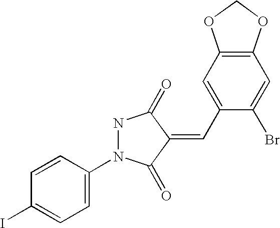 | 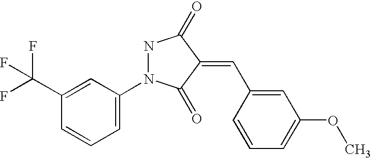 | 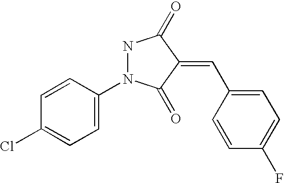 |
| 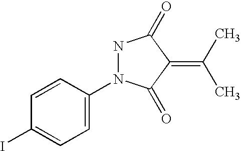 | 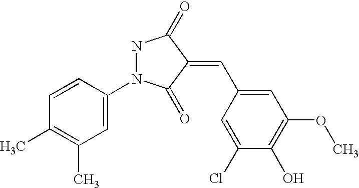 | 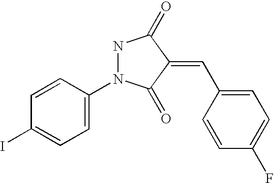 |
| 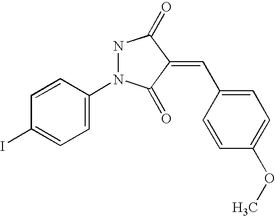 | 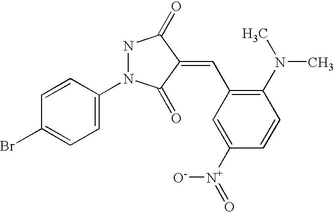 | 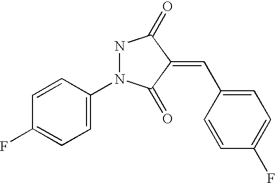 |
| 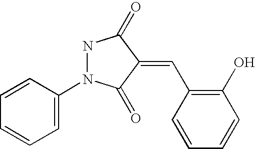 | 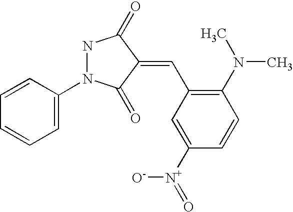 | 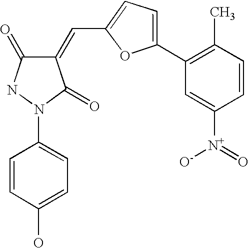 |
| 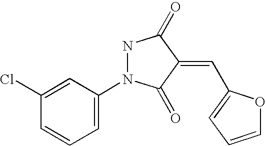 | 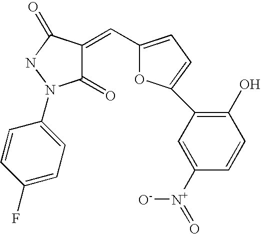 | 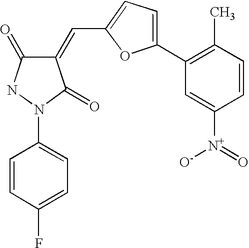 |
| 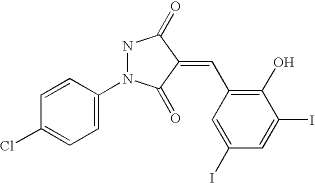 | 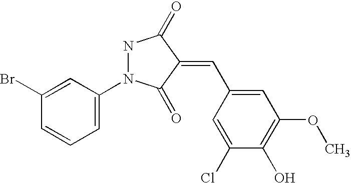 | 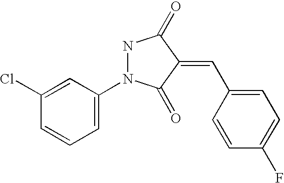 |
| 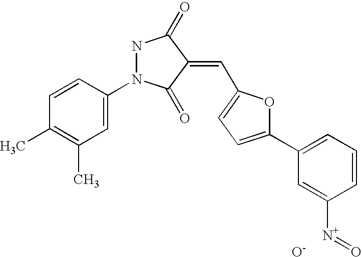 | 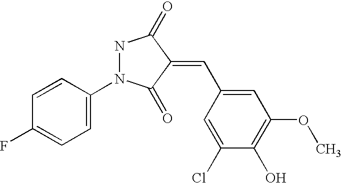 | 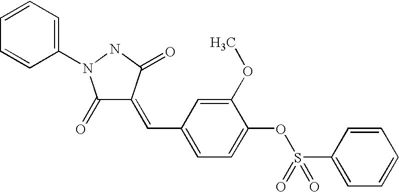 |
| 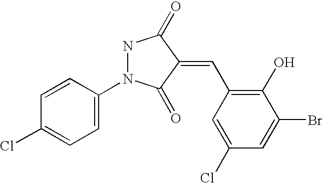 | 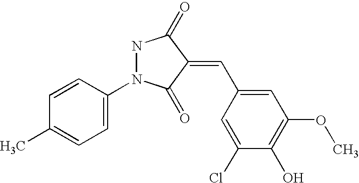 | 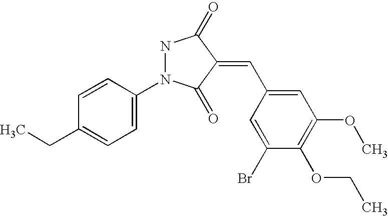 |
| 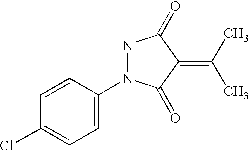 | 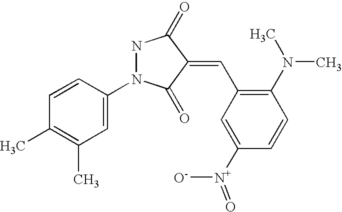 | 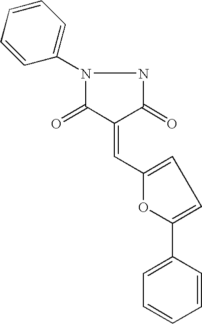 |
| 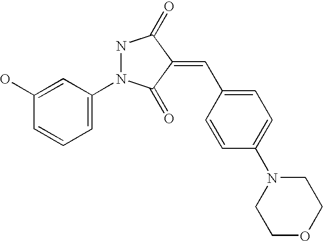 | 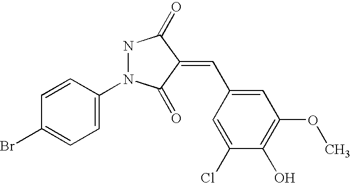 | 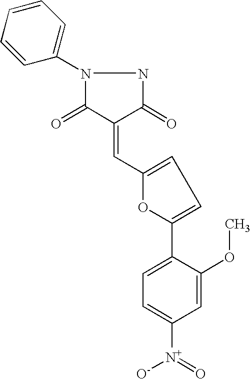 |
| 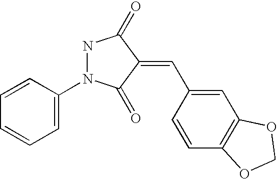 | 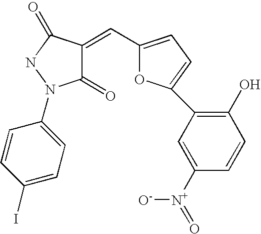 | 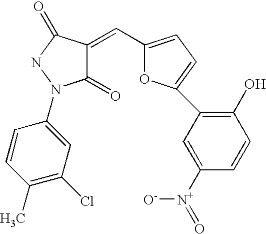 |
| 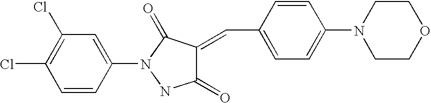 | 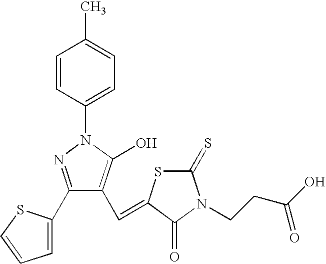 | 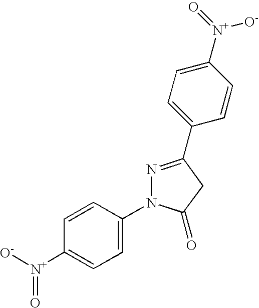 |
| 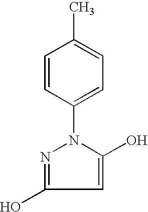 | 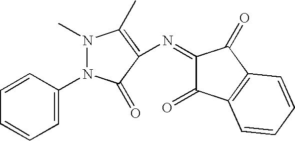 | |

TABLE 2

| | |
|---|---|
| 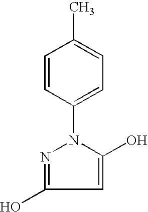 | 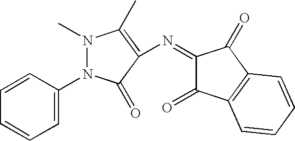 |
| 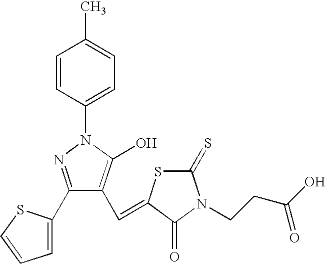 | 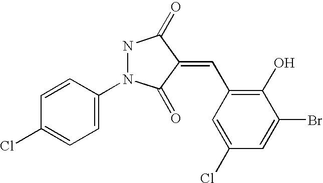 |
| 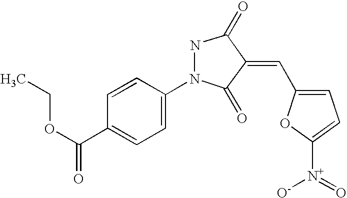 | 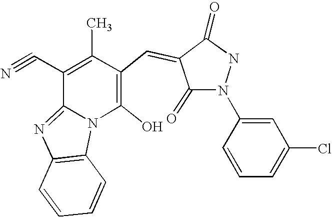 |
| 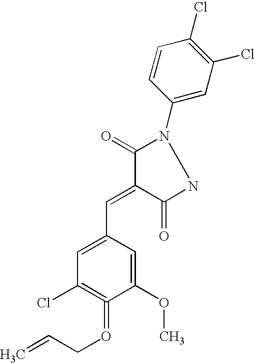 | 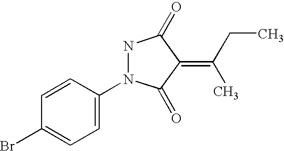 |
| 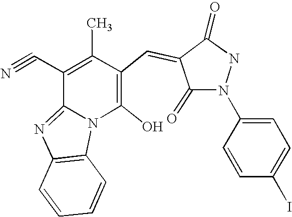 | 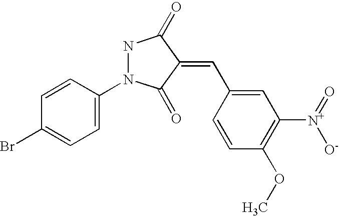 |
| 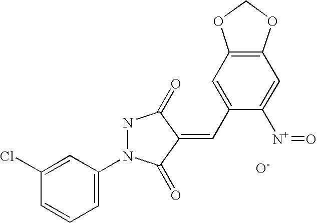 | 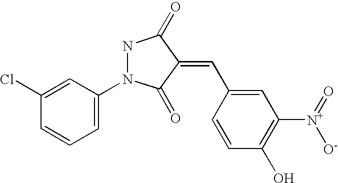 |
| 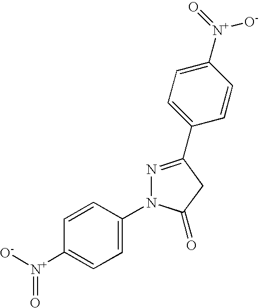 | |

In a particular embodiment of the invention, the Pin1 modulating compound of formula (I) is any one of the compounds of Table 1, or derivatives thereof. In certain embodiments, the Pin1 modulating compound of formula (I) is any one of the compounds of Table 2, or derivatives thereof.

In another embodiment, the invention pertains to the Pin1-modulating compounds of formula (I) described herein. Particular embodiments of the invention pertain to the modulating compounds of Table 1 or Table 2, or derivatives thereof.

In yet another embodiment, the invention pertains to pharmaceutical compositions comprising the Pin1-modulating compounds described herein and a pharmaceutical acceptable carrier.

The another embodiment, the invention is intended to include any novel compound described herein.

Additionally, the compounds described above are intended to include analogs containing art-recognized substituents that do not significantly effect the analog's ability to perform its intended function.

In an additional embodiment, the invention pertains, at least in part, to a method for treating cyclin D1 overexpression in a subject. This method includes administering to the subject an effective amount of a Pin1-modulating compound of formula (I), as described above, such that the cyclin D1 overexpression is treated. In certain embodiments, the overexpression of cyclin D1 is associated with the presence of breast cancer in the subject.

“Increased cyclin D1 expression” or “cyclin D1 overexpression” or “elevation in the expression of cyclin D1” includes cells having higher than normal levels of cyclin D1. Significant cyclin D1 overexpression includes both small and large increases in the levels of cyclin D1 compared with normal levels. Preferably, cyclin D1 overexpression is considered in the context of the phase of the cell cycle. In actively proliferating normal cells, cyclin D1 reaches a peak in mid G₁ phase, decreases during S-phase, and remains low throughout the rest of the cycle, however, in transformed cells the level of cyclin D1 is more variable. Therefore, cyclin D1 overexpression includes the expression of cyclin D1 at levels that are abnormally high for the particular cell cycle phase of the cell. Cyclin D1 overexpression can manifest itself as tumor growth or cancer. One skilled in the art would recognize the comparative studies that have been done measuring the level of cyclin D1 expression in normal cells in comparison with cells having a cancerous state.

Increased cyclin D1 expression has been found in a vast range of primary human tumors. Increased cyclin D1 expression has been detected in the form of gene amplification, increased cyclin D1 RNA expression, and increased cyclin D1 protein expression. Most clinical studies comparing cyclin D1 gene amplification with expression of cyclin D1 have found that more cases show over-expression of both RNA and protein than show amplification of the gene. The presence of increased cyclin D1 RNA and/or protein expression without gene amplification suggests that other cellular genes such as pRb may affect the expression cyclin D1. Human tumors found to have increased cyclin D1 expression include: parathyroid adenomas, mantle cell lymphomas, breast cancers, head and neck squamous cell carcinomas (i.e. squamous carcinomas in the oral cavity, nasopharynx, pharynx, hypopharynx, and larynx), esophageal cancers, hepatocellular carcinomas, colorectal cancers, genitourinary cancers, lung cancers (i.e. squamous cell carcinomas of the lung), skins cancers (i.e. squamous cell carcinomas, melanomas, and malignant fibrous histiocytomas), sarcomas, and central nervous system malignancies (i.e. astrocytomas and glioblastomas), gastric adenocarcinomas, pancreatic adenocarcinomas, squamous carcinomas of the gall bladder (Donnellan, et al. 1998. J Clin. Pathol: Mol. Pathol. 51:1-7). The cyclin D1 gene is amplified in approximately 20% of mammary carcinomas and the protein is overexpressed in approximately 50% of mammary carcinomas (Barnes, et al. 1998. Breast Cancer Research and Treatment. 52:1-15). Cyclin D1 overexpression in mantle cell lymphoma is discussed in Espinet, et al. 1999. Cancer Genet Cytogenet. 111(1):92-8 and Stamatopoulous, et al. 1999. Br. J Haematol. 105(1):190-7. Cyclin D1 overexpression in breast cancer is discussed in Fredersdorf, et al. 1997. PNAS 94(12):6380-5. Cyclin D1 overexpression in head and neck cancers is discussed in Matthias, et al. 1999. Cancer Epidemiol. Biomarkers Prev. 8(9):815-23; Matthias, et al. 1998. Clin. Cancer Res. 4(10):2411-8; and Kyomoto, et al. 1997. Int. J Cancer. 74(6):576-81. Cyclin D1 overexpression in laryngeal carcinoma is discussed in Bellacosa, et al. 1996. Clin. Cancer Res. 2(1):175-80. Cyclin D1 overexpression in multiple myeloma is discussed in Hoechtlen-Vollmar, et al. 2000. Br. J Haematol. 109(1):30-8; Pruneri, et al. 2000. Am. J Pathol. 156(5):1505-13; and Janssen, et al. 2000. Blood 95(8):2691-8. It is believed that in many tumors, cyclin D1 acts in co-operation with other oncogenes or tumor suppressor genes.

Cyclin D1 expression is regulated by many factors. Growth factors (i.e. CSF1, platelet-derived growth factor, insulin-like growth factor, steroid hormones, prolactin, and serum stimulation) promote the synthesis of cyclin D1 and removal of growth factors lead to a drop in cyclin D1 levels and arrest the cell in G₁. Hosokawa, et al. 1996. J Lab. Clin. Med. 127:246-52. While hypophosphorylated pRb stimulates cyclin D1 transcription, cyclin D1 activity is inhibited by transforming growth factor β-1, p53, and cyclin dependent kinase inhibitors (CKIs). High levels of CKIs bind to cdks and reduce the ability of cyclins to activate the cdks. The two classes of CKIs are the Kip/Cip family including p21, p27, and p57, capable of binding to and inhibiting most cyclin-cdk cornplexes, and the INK4 family including p15, p16, 18, and p19, which seem to be specific inhibitors of cyclin D1-cdk complexes. Donnellan, et al. 1998. J. Clin. Pathol: Mol. Pathol. 51:1-7. In addition, CKI p16 is activated by pRb and E2F, while the levels of CKI p27 are increased by TGF-β, cAMP, contact inhibition, and serum deprivation. Barnes, et al. 1998. Breast Cancer Research and Treatment. 52:1-15.

Cyclin D1 is believed to act through the phosphorylation of pRB, which is hypophosphorylated throughout the G₁ phase, phosphorylated just before the S phase, and remains phosphorylated until late mitosis. Hypophosphorylated pRB arrests cells in G₁ by forming a complex with the E2F family of DNA binding proteins that transcribe genes associated with DNA replication (the S phase of the cell cycle).

Cyclin D1 can form a complex with either cdk4 or cdk6 to form activated cdk4 or cdk6. Activated cdk4 or cdk6 induces the phosphorylation of pRb changing pRb from its hypophosphorylated form in which it binds to and inactivates E2F transcription factors to phosphorylated pRb that no longer binds to nor inactivates E2F transcription factors. In some mouse lymphoma cells overexpressing D cyclins, pRb is hyperphosphorylated compared with pRb in cells not overexpressing D cyclins. It appears that cyclin D1 is required to initiate the phosphorylation of pRb that, in turn, drives the cell through the restriction point at which stage the cell is committed to divide.

“Neoplasia” or “neoplastic transformation” is the pathologic process that results in the formation and growth of a neoplasm, tissue mass, or tumor. Such process includes uncontrolled cell growth, including either benign or malignant tumors. Neoplasms include abnormal masses of tissue, the growth of which exceeds and is uncoordinated with that of the normal tissues and persists in the same excessive manner after cessation of the stimuli that evoked the change. Neoplasms may show a partial or complete lack of structural organization and functional coordination with the normal tissue, and usually form a distinct mass of tissue. One cause of neoplasia is dysregulation of the cell cycle machinery.

Neoplasms tend to grow and function somewhat independently of the homeostatic mechanisms that control normal tissue growth and function. However, some neoplasms remain under the control of the homeostatic mechanisms that control normal tissue growth and function. For example, some neoplasms are estrogen sensitive and can be arrested by anti-estrogen therapy. Neoplasms can range in size from less than 1 cm to over 6 inches in diameter. A neoplasm even 1 cm in diameter can cause biliary obstructions and jaundice, if it arises in and obstructs the ampulla of Vater.

Neoplasms tend to morphologically and functionally resemble the tissue from which they originated. For example, neoplasms arising within the islet tissue of the pancreas resemble the islet tissue, contain secretory granules, and secrete insulin. Clinical features of a neoplasm may result from the function of the tissue from which it originated. For example, excessive amounts of insulin can be produced by islet cell neoplasms resulting in hypoglycemia which, in turn, results in headaches and dizziness. However, some neoplasms show little morphological or functional resemblance to the tissue from which they originated. Some neoplasms result in such non-specific systemic effects as cachexia, increased susceptibility to infection, and fever.

By assessing the histology and other features of a neoplasm, it can be determined whether the neoplasm is benign or malignant. Invasion and metastasis (the spread of the neoplasm to distant sites) are definitive attributes of malignancy. Despite the fact that benign neoplasms may attain enormous size, they remain discrete and distinct from the adjacent non-neoplastic tissue. Benign tumors are generally well circumscribed and round, have a capsule, and have a grey or white color, and a uniform texture. In contrast, malignant tumors generally have fingerlike projections, irregular margins, are not circumscribed, and have a variable color and texture. Benign tumors grow by pushing on adjacent tissue as they grow. As the benign tumor enlarges it compresses adjacent tissue, sometimes causing atrophy. The junction between a benign tumor and surrounding tissue may be converted to a fibrous connective tissue capsule allowing for easy surgical removal of the benign tumor.

Conversely, malignant tumors are locally invasive and grow into the adjacent tissues usually giving rise to irregular margins that are not encapsulated making it necessary to remove a wide margin of normal tissue for the surgical removal of malignant tumors. Benign neoplasms tend to grow more slowly and tend to be less autonomous than malignant tumors. Benign neoplasms tend to closely histologically resemble the tissue from which they originated. More highly differentiated cancers, i.e., cancers that resemble the tissue from which they originated, tend to have a better prognosis than poorly differentiated cancers, while malignant tumors are more likely than benign tumors to have an aberrant function, e.g., the secretion of abnormal or excessive quantities of hormones.

The histological features of cancer are summarized by the term “anaplasia.” Malignant neoplasms often contain numerous mitotic cells. These cells are typically abnormal. Such mitotic aberrations account for some of the karyotypic abnormalities found in most cancers. Bizarre multinucleated cells are also seen in some cancers, especially those that are highly anaplastic.

The term “anaplasia” includes histological features of cancer. These features include derangement of the normal tissue architecture, the crowding of cells, lack of cellular orientation termed dyspolarity, and cellular heterogeneity in size and shape termed “pleomorphism.” The cytologic features of anaplasia include an increased nuclear-cytoplasmic ratio (nuclear-cytoplasmic ratio can be over 50% for malignant cells), nuclear pleomorphism, clumping of the nuclear chromatin along the nuclear membrane, increased staining of the nuclear chromatin, simplified endoplasmic reticulum, increased free ribosomes, pleomorphism of mitochondria, decreased size and number of organelles, enlarged and increased numbers of nucleoli, and sometimes the presence of intermediate filaments.

The term “dysplasia” includes pre-malignant states in which a tissue demonstrates histologic and cytologic features intermediate between normal and anaplastic. Dysplasia is often reversible.

The term “cancer” includes malignancies characterized by deregulated or uncontrolled cell growth, for instance carcinomas, sarcomas, leukemias, and lymphomas. The term “cancer” includes primary malignant tumors, e.g., those whose cells have not migrated to sites in the subject's body other than the site of the original tumor, and secondary malignant tumors, e.g., those arising from metastasis, the migration of tumor cells to secondary sites that are different from the site of the original tumor.

The term “carcinoma” includes malignancies of epithelial or endocrine tissues, including respiratory system carcinomas, gastrointestinal system carcinomas, genitourinary system carcinomas, testicular carcinomas, breast carcinomas, prostate carcinomas, endocrine system carcinomas, melanomas, choriocarcinoma, and carcinomas of the cervix, lung, head and neck, colon, and ovary. The term “carcinoma” also includes carcinosarcomas, which include malignant tumors composed of carcinomatous and sarcomatous tissues. The term “adenocarcinoma” includes carcinomas derived from glandular tissue or a tumor in which the tumor cells form recognizable glandular structures.

The term “sarcoma” includes malignant tumors of mesodermal connective tissue, e.g., tumors of bone, fat, and cartilage.

The terms “leukemia” and “lymphoma” include malignancies of the hematopoietic cells of the bone marrow. Leukemias tend to proliferate as single cells, whereas lymphomas tend to proliferate as solid tumor masses. Examples of leukemias include acute myeloid leukemia (AML), acute promyelocytic leukemia, chronic myelogenous leukemia, mixed-lineage leukemia, acute monoblastic leukemia, acute lymphoblastic leukemia, acute non-lymphoblastic leukemia, blastic mantle cell leukemia, myelodyplastic syndrome, T cell leukemia, B cell leukemia, and chronic lymphocytic leukemia. Examples of lymphomas include Hodgkin's disease, non-Hodgkin's lymphoma, B cell lymphoma, epitheliotropic lymphoma, composite lymphoma, anaplastic large cell lymphoma, gastric and non-gastric mucosa-associated lymphoid tissue lymphoma, lymphoproliferative disease, T cell lymphoma, Burkitt's lymphoma, mantle cell lymphoma, diffuse large cell lymphoma, lymphoplasmacytoid lymphoma, and multiple myeloma.

For example, the therapeutic methods of the present invention can be applied to cancerous cells of mesenchymal origin, such as those producing sarcomas (e.g., fibrosarcoma, myxosarcoma, liosarcoma, chondrosarcoma, osteogenic sarcoma or chordosarcoma, angiosarcoma, endotheliosardcoma, lympangiosarcoma, synoviosarcoma or mesothelisosarcoma); leukemias and lymphomas such as granulocytic leukemia, monocytic leukemia, lymphocytic leukemia, malignant lymphoma, plasmocytoma, reticulum cell sarcoma, or Hodgkin's disease; sarcomas such as leiomysarcoma or rhabdomysarcoma, tumors of epithelial origin such as squamous cell carcinoma, basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, chorioaencinoma, semonoma, or embryonal carcinoma; and tumors of the nervous system including gioma, menigoma, medulloblastoma, schwannoma or epidymoma. Additional cell types amenable to treatment according to the methods described herein include those giving rise to mammary carcinomas, gastrointestinal carcinoma, such as colonic carcinomas, bladder carcinoma, prostate carcinoma, and squamous cell carcinoma of the neck and head region. Examples of cancers amenable to treatment according to the methods described herein include vaginal, cervical, and breast cancers.

The language “inhibiting undesirable cell growth” is intended to include the inhibition of undesirable or inappropriate cell growth. The inhibition is intended to include inhibition of proliferation including rapid proliferation. For example, the cell growth can result in benign masses or the inhibition of cell growth resulting in malignant tumors. Examples of benign conditions which result from inappropriate cell growth or angiogenesis are diabetic retinopathy, retrolental fibrioplasia, neovascular glaucoma, psoriasis, angiofibromas, rheumatoid arthritis, hemangiomas, Karposi's sarcoma, and other conditions or dysfunctions characterized by dysregulated endothelial cell division.

The language “inhibiting tumor growth” or “inhibiting neoplasia” includes the prevention of the growth of a tumor in a subject or a reduction in the growth of a pre-existing tumor in a subject. The inhibition also can be the inhibition of the metastasis of a tumor from one site to another. In particular, the language “tumor” is intended to encompass both in vitro and in vivo tumors that form in any organ or body part of the subject. The tumors preferably are tumors sensitive to the Pin1-modulating compounds of the present invention. Examples of the types of tumors intended to be encompassed by the present invention include those tumors associated with breast cancer, skin cancer, bone cancer, prostate cancer, liver cancer, lung cancer, brain cancer, cancer of the larynx, gallbladder, esophagus, pancreas, rectum, parathyroid, thyroid, adrenal, neural tissue, head and neck, colon, stomach, bronchi, kidneys. Specifically, the tumors whose growth rate is inhibited by the present invention include basal cell carcinoma, squamous cell carcinoma of both ulcerating and papillary type, metastatic skin carcinoma, osteo sarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neuromas, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multi forma, leukemias, lymphomas (i.e. maglinant lymphomas, mantle cell lymphoma), malignant melanomas, multiple mycloma, epidermoid carcinomas, and other carcinomas and sarcomas.

The Pin1 modulating compounds of the present invention may be used to treat, inhibit, and/or prevent undesirable cell growth, neoplasia, and/or cancer in any subject. The Pin1 modulating compounds of the present invention may be used to inhibit Pin1 activity in a subject. In one embodiment, the Pin1 modulating compounds of the present invention may be used to inhibit cyclin D1 expression in a subject.

In one embodiment, the invention pertains, at least in part, to a method for treating a Pin1-associated state in a subject. The method includes administering to a subject an effective amount of a combination of a Pin1 modulating compound of the invention, e.g., Pin1-modulating compounds of formula (I) as described above, and a hyperplastic inhibitory agent to treat the Pin1 associated states.

In another embodiment, the invention pertains, at least in part, to a method for treating cyclin D1 overexpression in a subject. The method includes administering to a subject an effective amount of a combination of a Pin1 modulating compound of the invention, e.g., Pin1-modulating compounds of formula (I) as described above, and a hyperplastic inhibitory agent to treat the cyclin D1 overexpression.

In yet another embodiment, the invention pertains, at least in part, to a method for treating cancer in a subject. The method includes administering to a subject an effective amount of a combination of a Pin1 modulating compound of the invention, e.g., Pin 1-modulating compounds of formula (I) as described above, and a hyperplastic inhibitory agent to treat the cancer.

The language “hyperplastic inhibitory agent” includes agents that inhibit the growth of proliferating cells or tissue wherein the growth of such cells or tissues is undesirable. For example, the inhibition can be of the growth of malignant cells, such as in neoplasms or benign cells, e.g., in tissues where the growth is inappropriate. Examples of the types of agents that can be used include chemotherapeutic agents, radiation therapy treatments, including therapeutically effective ranges of light, e.g., laser light and/or immunofluorescent compounds, and associated radioactive compounds and methods, immunotoxins, and combinations thereof.

The language “chemotherapeutic agent” includes chemical reagents that inhibit the growth of proliferating cells or tissues wherein the growth of such cells or tissues is undesirable. Chemotherapeutic agents are well known in the art (see e.g., Gilman A. G., et al., The Pharmacological Basis of Therapeutics, 8th Ed., Sec 12:1202-1263 (1990)), and are typically used to treat neoplastic diseases. The chemotherapeutic agents generally employed in chemotherapy treatments are listed below in Table 3. Other similar examples of chemotherapeutic agents include: bleomycin, docetaxel (Taxotere), doxorubicin, edatrexate, etoposide, finasteride (Proscar), flutamide (Eulexin), gemcitabine (Gemzar), goserelin acetate (Zoladex), granisetron (Kytril), irinotecan (Campto/Camptosar), ondansetron (Zofran), paclitaxel (Taxol), pegaspargase (Oncaspar), pilocarpine hydrochloride (Salagen), porfimer sodium (Photofrin), interleukin-2 (Proleukin), rituximab (Rituxan), topotecan (Hycamtin), trastuzumab (Herceptin), tretinoin (Retin-A), Triapine, vincristine, and vinorelbine tartrate (Navelbine).

TABLE 3

| | | NONPROPRIETARY |
|---|---|---|
| | | NAMES |
| CLASS | TYPE OF AGENT | (OTHER NAMES) |
| Alkylating | Nitrogen Mustards | Mechlorethamine (HN₂) |
| | | Cyclophosphamide |
| | | Ifosfamide |
| | | Melphalan |
| | | (L-sarcolysin) |
| | | Chlorambucil |
| | Ethylenimines | Hexamethylmelamine |
| | And Methylmelamines | Thiotepa |
| | Alkyl Sulfonates | Busulfan |
| | Nitrosoureas | Carmustine (BCNU) |
| | | Lomustine (CCNU) |
| | | Semustine |
| | | (methyl-CCNU) |
| | | Streptozocin |
| | | (streptozotocin) |
| | Triazenes | Decarbazine (DTIC; |
| | | dimethyltriazenoimi- |
| | | dazolecarboxamide) |
| | Alkylator | cis-diamminedi- |
| | | chloroplatinum |
| | | II (CDDP) |
| Antimetabolites | Folic Acid Analogs | Methotrexate |
| | | (amethopterin) |
| | Pyrimidine | Fluorouracil (′5- |
| | Analogs | fluorouracil; 5-FU); |
| | | Floxuridine (fluorode- |
| | | oxyuridine); Fudr |
| | | Cytarabine (cyosine |
| | | arabinoside) |
| | Purine Analogs | Mercaptopuine (6- |
| | and Related | mercaptopurine; |
| | Inhibitors | 6-MP) Thioguanine |
| | | (6-thioguanine; TG) |
| | | Pentostatin (2′- |
| | | deoxycoformycin) |
| Natural Products | Vinca Alkaloids | Vinblastin (VLB) |
| | | Vincristine |
| | Topoisomerase | Etoposide |
| | Inhibitors | Teniposide |
| | | Camptothecin |
| | | Topotecan |
| | | 9-amino-campotothecin |
| | | CPT-11 |
| | Antibiotics | Dactinomycin |
| | | (actinomycin D) |
| | | Adriamycin |
| | | Daunorubicin |
| | | (daunomycin; |
| | | rubindomycin) |
| | | Doxorubicin |
| | | Bleomycin |
| | | Plicamycin |
| | | (mithramycin) |
| | | Mitomycin |
| | | (mitomycin C) |
| | | Taxol |
| | | Taxotere |
| | Enzymes | L-Asparaginase |
| | Biological Response | Interfon alfa |
| | Modifiers | Interleukin 2 |
| Miscellaneous | Platinum Coordination | cis-diamminedi- |
| Agents | Complexes | chloroplatinum |
| | | II (CDDP) |
| | | Carboplatin |
| | Anthracendione | Mitoxantrone |
| | Substituted Urea | Hydroxyurea |
| | Methyl Hydraxzine | Procarbazine |
| | Derivative | (N-methylhydrazine, |
| | | (MIH) |
| | Adrenocortical | Mitotane |
| | Suppressant | (o,p′-DDD) |
| | | Aminoglutethimide |
| Hormones and | Adrenocorticosteroids | Prednisone |
| Antagonists | | |
| | Progestins | Hydroxyprotesterone |
| | | caproate |
| | | Medroxyprogesterone |
| | | acetate |
| | | Megestrol acetate |
| | Estrogens | Diethylstilbestrol |
| | | Ethinyl estradiol |
| | Antiestrogen | Tamoxifen |
| | Androgens | Testosterone |
| | | propionate |
| | | Fluoxymesterone |
| | Antiandrogen | Flutamide |
| | Gonadotropin-releasing | Leuprolide |
| | Hormone analog | |

The language “radiation therapy” includes the application of a genetically and somatically safe level of electrons, protons, or photons, both localized and non-localized, to a subject to inhibit, reduce, or prevent symptoms or conditions associated with undesirable cell growth. The term X-rays is also intended to include machine-generated radiation, clinically acceptable radioactive elements, and isotopes thereof, as well as the radioactive emissions therefrom. Examples of the types of emissions include alpha rays, beta rays including hard betas, high-energy electrons, and gamma rays. Radiation therapy is well known in the art (see e.g., Fishbach, F., Laboratory Diagnostic Tests, 3rd Ed., Ch. 10:581-644 (1988)), and is typically used to treat neoplastic diseases.

The term “immunotoxins” includes immunotherapeutic agents that employ cytotoxic T cells and/or antibodies, e.g., monoclonal, polyclonal, phage antibodies, or fragments thereof, which are utilized in the selective destruction of undesirable rapidly proliferating cells. For example, immunotoxins can include antibody-toxin conjugates (e.g., Ab-ricin and Ab-diptheria toxin), antibody-raiiolabels (e.g., Ab-I¹³⁵) and antibody activation of the complement at the tumor cell. The use of immunotoxins to inhibit, reduce, or prevent symptoms or conditions associated with neoplastic diseases are well known in the art (see, e.g., Harlow, E. and Lane, D., Antibodies, (1988)).

In one embodiment, the invention includes a packaged Pin1-associated state treatment. The packaged treatment includes a Pin1 modulating compound of the invention, e.g., Pin1-modulating compounds of formula (I) as described above, packaged with instructions for using an effective amount of the Pin1 modulating compound.

In another embodiment, the invention includes a packaged cyclin D1 overexpression treatment. This packaged treatment include a Pin1 modulating compound of the invention, e.g., Pin1-modulating compounds of formula (I) as described above, packaged with instructions for using an effective amount of the Pin1 modulating compound to treat cyclin D1 overexpression.

In yet another embodiment, the invention also pertains, at least in part to a packaged cancer treatment, which includes a Pin 1-modulating compound of the invention, e.g., Pin1-modulating compounds of formula (I) as described above, packaged with instructions for using an effective amount of the Pin1-modulating compound to treat cancer.

The invention also pertains, at least in part, to pharmaceutical compositions of comprising Pin1-modulating compounds of the invention, e.g., Pin1-modulating compounds of formula (I) as described above, and, optionally, a pharmaceutically acceptable carrier.

The language “effective amount” of the compound is that amount necessary or sufficient to treat or prevent a Pin1 associated state, e.g. prevent the various morphological and somatic symptoms of a Pin1 associated state. In an example, an effective amount of the Pin1-modulating compound is the amount sufficient to inhibit undesirable cell growth in a subject. In another example, an effective amount of the Pin1-modulating compound is the amount sufficient to reduce the size of a pre-existing benign cell mass or malignant tumor in a subject. The effective amount can vary depending on such factors as the size and weight of the subject, the type of illness, or the particular Pin1 binding compound. For example, the choice of the Pin1 binding compound can affect what constitutes an “effective amount”. One of ordinary skill in the art would be able to study the factors contained herein and make the determination regarding the effective amount of the Pin1 binding compound without undue experimentation. In one possible assay, an effective amount of a Pin1-modulating compound can be determined by assaying for the expression of cyclin D1 and determining the amount of the Pin1-modulating compound sufficient to reduce the levels of cyclin D1 to that associated with a non-cancerous state.

The regimen of administration can affect what constitutes an effective amount. The Pin1 binding compound can be administered to the subject either prior to or after the onset of a Pin1 associated state. Further, several divided dosages, as well as staggered dosages, can be administered daily or sequentially, or the dose can be continuously infused, or can be a bolus injection. Further, the dosages of the Pin1 binding compound(s) can be proportionally increased or decreased as indicated by the exigencies of the therapeutic or prophylactic situation.

The language “pharmaceutical composition” includes preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier.

The phrase “pharmaceutically acceptable carrier” is art recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible-substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical, transdermal, buccal, sublingual, rectal, vaginal and/or parenteral administration. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect. Generally, out of one hundred per cent, this amount will range from about 1 per cent to about ninety-nine percent of active ingredient, preferably from about 5 per cent to about 70 per cent, most preferably from about 10 per cent to about 30 per cent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluent commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert dilutents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the active compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

The preparations of the present invention may be given orally, parenterally, topically, or rectally. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administration is preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound that is the lowest dose effective to produce a therapeutic effect. Such an effective dose will generally depend upon the factors described above. Generally, intravenous and subcutaneous doses of the compounds of this invention for a patient, when used for the indicated analgesic effects, will range from about 0.0001 to about 100 mg per kilogram of body weight per day, more preferably from about 0.01 to about 50 mg per kg per day, and still more preferably from about 1.0 to about 100 mg per kg per day. An effective amount is that amount treats an Pin1 associated state.

If desired, the effective daily dose of the active compound may be administered as two, three, four, five, six or more sub-doses administered separately at appropriate intervals throughout the day, optionally, in unit dosage forms.

While it is possible for a compound of the present invention to be administered alone, it is preferable to administer the compound as a pharmaceutical composition.

### EXEMPLIFICATION OF THE INVENTION

The invention is further illustrated by the following examples, which should not be construed as farther limiting. The animal models used throughout the Examples are accepted animal models and the demonstration of efficacy in these animal models is predictive of efficacy in humans.

### Tumor Inhibition Assays

Pin1-modulating compounds are potent antitumor agents. The anti-tumor activity of Pin1-modulating compounds against glioblastoma cells is comparable to 1,3-bis(2-chloroethyl)-1-nitrosourea (BCNU), one of the most potent clinical useful antitumor agents. Misra, et al. 1982. J. Am. Chem. Soc. 104:4478-4479

In vitro anti-tumor activity of Pin1-modulating compounds can be assayed by measuring the ability of Pin1-modulating compounds to kill tumor cells. Examples of appropriate cells lines include: human lung (A549); resistant human lung with low topo II activity (A549-VP); murine melanoma (B16); human colon tumor (HCT116); human colon tumor with elevated p170 levels (HCTVM); human colon tumor with low topo II activity (HCTVP); P388 murine lymph leukemia cells; and human colon carcinoma cell line (Moser) under standard conditions. After the cells are cultured for twenty-four hours and allowed to attach to a plate (i.e. a 96-well flat bottom plate), the cells are incubated for 72 hours with serially diluted concentrations of Pin1-modulating compounds. From this data, the concentration of the compound at which 50% of the cells are killed (IC₅₀) is determined. Kelly, et al., U.S. Pat. No. 5,166,208 and Pandey, et. al. 1981. J Antibiot. 34(11):1389-401.

In vivo anti-tumor activity of Pin1-modulating compounds can be assayed for by a reduction of tumor cells in mammals (i. e. mice) and a resulting increase in survival time compared to untreated tumor bearing mammals. For example, CDF₁ mice are injected interperitoneally with a suspension of P388 murine lymph leukemia cells, Ehrlich carcinoma cells, B16 melanoma cells, or Meth-A fibrosarcoma cells or other appropriate tumor cell line. Some of the mice are treated intraperitoneally with a Pin1-modulating compounds. Other mice are treated with saline. The in vivo activity of the compound is determined in terms of the % T/C which is the ratio of the mean survival time of the treated group to the mean survival time of the saline treated group times 100. Yokoi, et al, U.S. Pat. No. 4,584,377; Kelly, et al., U.S. Pat. No. 5,166,208; Wamick-Pickle, et al. 1981. J Antibiot. 34(11):1402-7; and Pandey, et. al. 1981. J Antibiot. 34(11):1389-401

The in vivo anti-tumor activity of Pin1-modulating compounds can also be assayed as inhibitors against an ovarian tumor growing in a human tumor cloning system. Tebbe, et al. 1971 J Am. Chem. Soc. 93:3793-3795.

The invention is further illustrated by the following examples, which should not be construed as further limiting.

### EXAMPLE 1

### Cell Based Cytotoxicity Assay (CBCA) of Pin1 Modulating Compounds

Mammalian cells are seeded in 96 well flat bottom microtiter plates at a density of 5,000 6000 cells per well on day 0 in 0.1 mL of an appropriate growth media. On Day 1, the wells are aspirated and 0.1 mL of fresh media is added. The cells are then treated with 0.01 mL of 10×drug dilutions in 10% DMSO in media and incubated at 37° C. in a humidified, 5% CO₂ atmosphere. The assay contained eight drug concentrations in triplicate as well as a triplicate control where cells are treated with 0.01 mL of 10% DMSO in media. On Day 4, the cells are incubated with 0.02 mL of a colorimetric cell-viability assay solution (MTS) prepared from 20 parts (3-(4,5-dimethylthiazol-2-yl)-5-(3-carboxymethoxyphenyl)-2-(4-sulfophenyl)-2H-tetrazolium (Promega) at 2.0 mg/mL in PBS and 1 part phenazine methosulfate (Sigma) at 0.92 mg/mL in PBS for 2-3 hours at 37° C. Background wells are prepared by incubating 0.02 mL of the colorimetric cell-viability assay solution with 0.1 mL of media in parallel with the cell containing wells. The absorbance at 490 nm is then measured with an ELISA plate reader and the absorbance is recorded for the background wells is averaged and the mean value is subtracted from the cell containing wells. Percent cell viabilities at each drug concentration are calculated by dividing the mean absorbance at 490 nm of the treated wells by the mean absorbance at 490 nm of the untreated wells. EDso values (the effective dose required to for 50% viability) are calculated by plotting drug concentrations versus percent cell viability.

To count cells, suspended cells (0.02 mL) are diluted into 0.18 mL of 0.2% trypan blue solution in PBS. Approximately 0.015 mL of the suspension is added to a chamber of a Levy counting hemacytometer. The viable cells are counted in each of the four sets of 16 squares that are at the corners of the closely ruled lines. The total number of viable cells from the 64 squares are then multiplied by 0.025 to obtain the concentration of cells in the stock suspension. (Number of cells in the 64 wells)×(0.025)=×10⁶ cells/mL (original stock).

### EXAMPLE 2

### Specificity Assay for Inhibition of Proline Isomerase by Pin1 Modulating Compounds

The proline isomerase activity assay is based on the method described by Fisher et al. (Biomed. Biochim. Acta, 1984, 43: 1101-1111). Specifically, the enzyme (112 ng) was preincubated with 72 mM substrate at 4° C. for 30 minutes in an 80 μL reaction volume containing 0.02 mg/μL BSA, 0.8 mM DTT, and 35 mM HEPES (pH 7.8). Proteolysis of the substrate was initiated by the addition of 80 μL of trypsin at 0.4 mg/mL in 35 mM HEPES (pH 7.8) and the release of p-nitroaniline was monitored every 10 seconds at 390 nm using a microplate reader (MRD/8V/DIAS, Dynex Technologies). Inhibition studies were preformed by adding 5 μL of inhibitors added in the pre-incubation mix. Inhibitors were at 0.4 mg/mL in 10% DMSO.

Multiple activity-based assays at multiple dilutions, performed as described above, were used to generate the curve from which the IC₅₀ was determined. Several IC₅₀ results were obtained for compounds in Table 2 using this experimental protocol.

### EXAMPLE 3

### Specificity Assay for Inhibition of Pin1 by Pin1 Modulating Compounds

The specificity of the Pin1 inhibitor compounds of the invention can be determined by the protease-coupled PPIase assay developed by Fischer et al. (Biomed. Biochim. Acta, 1984, 43: 1101-1111). For example, the enzyme activity of Pin1 can be compared to members of the other known classes of PPIases, cyclophilins (e.g., hCyp18, hCyP-A, hCyP-B, hCyP-C, and NKCA) and FKBPs (e.g., hFKBP12, hFKBP-12, hFKBP-13, and hFKBP-25) in the presence and absence of the compound.

In one assay, hPin1 activity measurements are determined using bovine trypsin (final concentration 0.21 mg/mL, Sigma) as an isomer specific protease and Ac-Ala-Ala-Ser(P)-Pro-Arg-pNA (Jerini, Germany) as a substrate. PPIase activity of hFKBP12 (Sigma) and hCyp18 (Sigma) is determined with the peptide substrate Suc-Ala-Phe-Pro-Phe-pNA (Bachem) and the protease α-chymotrypsin (final concentration 0.41 mg/mL, Sigma). The test can be performed by observing the released 4-nitroanilide at 390 nm with a Hewlett-Packard 8453 UV-vis spectrophotometer at 10° C. The total reaction volume is adjusted to 1.23 mL by mixing appropriate volumes of 35 mM HEPES (pH 7.8) with enzyme and effector solutions. The Pin1 inhibitor compound is freshly diluted from a 1 mg/mL stock solution in DMSO, and pre-incubated at varying concentrations with the enzyme for 5 min (10° C.). Prior to the start of reaction by addition of the respective protease, 2 μL of the peptide substrate stock solution (10 mg/mL in DMSO) is added. The amount of organic solvent is kept constant within each experiment (<0.1%). The pseudo-first-order rate constant k_{obs} for cis/trans isomerization in the presence of PPIase and the first-order rate constant k₀ of the uncatalyzed cis/trans isomerization can be calculated using the Kinetics Software of Hewlett-Packard as well as SigmaPlot2000 for Windows 6.0 (SPSS).

The Kᵢ value for inhibition of Pin1 PPIase activity by a Pin1 inhibitor compound of the invention at constant concentrations of substrate ([S₀]<<K_{M}) can then be calculated by fitting the data according to the equation for a competitive “tight-binding” inhibitor using SigmaPlot2000

### EXAMPLE 4

### Cellular Screen Secondary Cell Based Activity Assay (Determination of ED₅₀)

A cell solution is added to a flask containing containing 13 ml of 10% FBS with EDTA. The cell suspension is centrifuged at 1500 g for 5 minutes and resuspend in 10 mL media. The centriguge procedure is repeated. The cells are resuspended in 2 mL of media. 20 μl of cell suspension is added to 180 μL 0.2% trypahn blue. Approximately 2000 cells are added to each well of a micortitre plate in 100 μL media.

After cells have grown for an appropriate time (˜1-2 days depending on cell line) 10 μL of a stock solution containing a test compound is added to each well. After further growth, the media is removed from the well and tyrpsin is added. After a short incubation, the trypsin is removed or inactivated and the cells are counted using a Guava Cell Analysis System (Hayward, Calif.).

### EXAMPLE 5

### Method for Evaluating Pin1 Levels

In one embodiment, the automated cellular imaging system (ACIS) was used to determine tissues with elevated Pin1 Levels. The data that is presented in Example 4 is from U.S. patent application Ser. No. 10/071,747, filed Feb. 8, 2002, the entire contents of which are incorporated by reference.

Micro-histoarray sections were scanned and images were captured using the automated cellular imaging system (ChromaVision Medical Systems, Inc., San Juan Capistrano, Calif.), which combines automated microscopy and computerized image processing to analyze multiple tissues on a single slide. ACIS was used to analyze microarray tissue sections on glass slides stained using a diaminodenzidine chromagen (DAB) and hematoxylin counterstain. Positive staining (brown color) as viewed by light microscope indicates the presence of the protein, and color intensity correlates directly with protein quantity (expression). The ACIS was able to recognize 255 levels of immnohistochemical staining intensity (0-255) and converted these to fractional scores for the selected individual areas. However, the base limit on the threshold for the Generic DAB is pre-set at 50 by the manufacturer because the system is very sensitive. Therefore, any intensity below 50 was treated as 0 in this study. Entire immunostained tissue sections were scanned using the 4×objective and images were captured using the 10×objective.

Calculation of Pin Protein Expression in Human Cancers

In this study, intensity scoring and the percent positive scoring (brown area was divided by total area) were used with the entire individual tissue dot selected. The immunohistochemical staining was quantitated without knowledge of a pathologist's score. All tissue samples were immunostained twice at one location, e.g., the University of Basel and confirmed at a second location, e.g., Pintex Pharmaceuticals, Inc., followed by an evaluation of the two data sets, e.g., at Pintex Pharmaceuticals, Inc. For example, the final score was obtained by using the average of the two data sets and was calculated by the formulation:

score=intensity+(X percent positive staining).

The % of total cases showing elevated levels (over-expression) of Pin1=

[numbers of tumor samples with score larger than the score of the highest normal case]total number of tumor samples multiplied by 100.

Results

TABLE 4
Pin1 protein over-expression in human tissues microarray

| | | % of Tumor Cases |
|---|---|---|
| | Case | with Eleveted |
| Tumor type | number | Level of Pin1 |
| Brain tumor (3) | 111 | |
| Oligodendroglioma | 20 | 90 |
| Astrocytoma | 46 | 63 |
| Glioblastomamultiforme | 45 | 87 |
| Genecological tumor (13) | 372 | |
| Cervical carcinoma | 42 | 81 |
| Endometrium, endometroid carcinoma | 46 | 0 |
| Endometrium, serous carcinoma | 13 | 0 |
| Ovary, endometroid cancer | 45 | 24 |
| Ovary, Brenner tumor | 8 | 63 |
| Ovary mucinous cancer | 12 | 58 |
| Ovary, serous cancer | 47 | 43 |
| Uterus, carcinosarcoma | 6 | 100 |
| Breast, lobular cancer | 36 | 56 |
| Breast, ductal cancer | 47 | 47 |
| Breast, medullary cancer | 24 | 29 |
| Breast, mucinous cancer | 24 | 29 |
| Breast tubular cancer | 22 | 9 |
| Endocrine tumor (8) | 213 | |
| Thyroid adenocarcinoma | 42 | 29 |
| Thyroid follicular cancer | 49 | 41 |
| Thyroid medullary cancer | 8 | 100 |
| Thyroid papillary car | 36 | 22 |
| Parathyroid, adenocarcinoma | 28 | 21 |
| Adrenal gland adenoma | 15 | 0 |
| Adrenal gland cancer | 6 | 33 |
| Pheochromocytoma | 29 | 0 |
| Digestive tract tumor (11) | 411 | |
| Colon adenoma mild displasia | 47 | 21 |
| Colon adenoma moderate displasia | 47 | 17 |
| Colon adenoma severe displasia | 49 | 14 |
| Colon adenocarcinoma | 43 | 2 |
| Esophagus adenocarcinoma | 43 | 30 |
| Hepatocelluar carcinoma | 34 | 62 |
| Mouth cancer | 46 | 93 |
| Gall bladder adenocarcinoma | 28 | 14 |
| Pancreatic adenocarcinoma | 43 | 2 |
| Small intestine adenocarcinoma | 10 | 0 |
| Stomach diffuse adenocarcinoma | 21 | 0 |
| Genitourinary tract tumor (9) | 381 | |
| Prostate (hormone-refract) | 44 | 59 |
| Prostate (untreated) | 47 | 64 |
| Kidney chromophobic carcinoma | 15 | 0 |
| Kidney clear cell carcinoma | 47 | 0 |
| Kidney oncocytoma | 8 | 0 |
| Kidney papillary carcinoma | 44 | 0 |
| Testis, non-seminomatous cancer | 43 | 2 |
| Testis seminoma | 47 | 2 |
| Urinary bladder transitional | 86 | 2 |
| carcinoma | | |
| Respiratory tract tumor (4) | 184 | |
| Lung, adenocarcinoma | 44 | 27 |
| Lung, large cell cancer | 45 | 42 |
| Lung, small cell cancer | 47 | 57 |
| Lung, squmous cell carcinoma | 48 | 44 |
| Hematological neoplasia (5) | 146 | |
| Hodgkin lymphoma | 23 | 0 |
| MALT lymphoma | 47 | 4 |
| NHL, diffuse large B | 22 | 18 |
| NHL, others | 30 | 23 |
| Thymoma | 24 | 8 |
| Skin tumor (5) | 178 | |
| Skin, malignant melanoma | 44 | 73 |
| Skin, basolioma | 44 | 39 |
| Skin, squamous cell cancer | 39 | 13 |
| Skin, merkel zell cancer | 5 | 100 |
| Skin benign nevus | 46 | 52 |
| Soft tissue tumor (2) | 45 | |
| Lipoma | 25 | 20 |
| Liposarcoma | 20 | 75 |

Equivalents

Those skilled in the art will recognize, or be able to ascertain using no more than routine experimentation, many equivalents to the specific embodiments and methods described herein. Such equivalents are intended to be encompassed by the scope of the following claims.

Incorporation by Reference

The entire contents of all patents, published patent applications and other references cited herein are hereby expressly incorporated herein in their entireties by reference.

1. A method for treating a Pin1-associated state in a subject comprising administering to said subject an effective amount of a Pin1-modulating compound of formula (I): 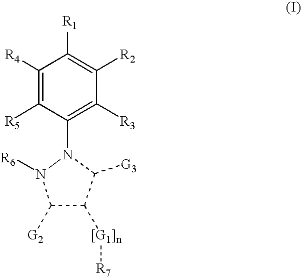
wherein
the dashed lines indicate a single or a double bond;
n is 0, 1, or 2;
G₁ is CH₂, CH, or N;
G₂ and G₃ are independently selected from the group consisting of O, OR₂, NR₂, NR₂R₆, hydrogen, alkyl, aryl;
R₁, R₂, R₃, R₄, R₅, R₆ and R₇ are each independently substituted or unsubstituted: alkyl, alkenyl, alkynyl, aryl, hydrogen, acyl, or any combination thereof;
such that said Pin1-associated state is treated.

2. The method of claim 1, wherein G₂ is O, hydrogen, CH₃, thiophene, or nitrophenyl; G₃ is O, H, or OH; and R₆ is H or CH₃.

3. The method of claim 1, wherein R₇ is a phenyl, a pyridine, an indole, an indene, a pyrazole, a benzoimidazole, a thiophene, a naphthalene, a morpholine, a pyrrolidine, a piperidine, a furan, a tetrahydrofuran, a benzo[1,3]dioxole, a pyrole, a cyclohexene, a furazan-2-oxide, a thioxo-thiazolidinone, an isobenzofuranone, a benzo[4,5]imidazo[1,2-a]pyridine, an isobutyl, a derivative thereof, or
a combination thereof, wherein the combination may further comprise alkyl, alkenyl, alkynyl, or acyl groups.

4. The method of claim 3, wherein R₇ substituted with substituents selected from the group consisting of H, O, OH, Cl, Br, F, I, OEt, OMe, CO₂H, propenyloxy, acetyl, isopropyl, butyloxy, benzyloxy, propyloxy, morpholino, dimethylamino, NO₂, sulfonamide, CO₂CH₂CH₃, —OCH₂CO₂CH₂CH₃, benzene sulfonate, acetamide, Me, t-butyl, propargyl, naphthyl, naphthyloxy, propargyloxy, hexyloxy, octyloxy, dipropylamino, ethylmethylamino, propyloxy, iso-propyl, benzyl, phenyl, methylsulfanyl, phenylsulfanyl, naphthylsulfanyl, benzoyl, , CO₂CH₃, —OCH₂CO₂CH₃, —OCH₂CO₂H, —OCH₂CH₂O—R₃, —CH₂CH₂O—R₃, —OCH₂CH₂S—R₃, derivatives thereof, and combinations thereof.

5. The method of claim 1, wherein R₁, R₂, R₃, R₄, and R₅ are each independently selected from the group consisting of CH₃, Br, I, Cl, H, F, CO₂CH₂CH₃, CF₃, Et, combinations thereof, and derivatives thereof.

6. The method of claim 1, wherein said Pin1-modulating compound is a Pin1-inhibiting compound.

7. The method of claim 1, wherein said compound is selected from the group consisting of compounds listed in Table 1, and derivatives thereof.

8. The method of claim 1, wherein said compound is selected from the group consisting of compounds listed in Table 2, and derivatives thereof.

9. The method of claim 1, wherein said Pin1-associated state is a cyclin D1 elevated state.

10. The method of claim 1, wherein said Pin1-associated state is neoplastic transformation.

11. The method of claim 1, wherein said Pin1-associated state is cancer.

12. The method of claim 1, wherein said Pin1-associated state is tumor growth.

13. The method of claim 1, wherein said method of treating said Pin1-associated state comprises inhibiting tumor growth.

14. The method of claim 1, wherein said method of treating said Pin1-associated state comprises preventing the occurrence of tumor growth in the subject.

15. The method of claim 1, wherein said method of treating said Pin1-associated state comprises reducing the growth of a pre-existing tumor in the subject.

16. The method of claim 1, wherein said Pin1-associated state is colon cancer or breast cancer.

17. The method of claim 1, wherein said Pin1-associated state is sarcoma or a malignant lymphoma.

18. The method of claim 1, wherein said Pin1-associated state is esophageal cancer, oligodendroglioma, astrocytoma, glioblastomamultiforme, cervical carcinoma, ovary endometroid cancer, ovary Brenner tumor, ovary mucinous cancer, ovary serous cancer, uterus carcinosarcoma, breast lobular cancer, breast ductal cancer, breast medullary cancer, breast mucinous cancer, breast tubular cancer, thyroid adenocarcinoma, or thyroid follicular cancer.

19. The method of claim 1, wherein said Pin1-associated state is thyroid medullary cancer, thyroid papillary carcinoma, parathyroid adenocarcinoma, adrenal gland adenoma, adrenal gland cancer, pheochromocytoma, colon adenoma mild displasia, colon adenoma moderate displasia, colon adenoma severe displasia, or colon adenocarcinoma.

20. The method of claim 1, wherein said Pin1-associated state is esophagus adenocarcinoma, hepatocelluar carcinoma, mouth cancer, gall bladder adenocarcinoma, pancreatic adenocarcinoma, prostate, prostate cancer, testis non-seminomatous cancer, testis seminoma, urinary bladder transitional carcinoma, lung adenocarcinoma, lung large cell cancer, lung small cell cancer, lung squamous cell carcinoma, MALT lymphoma, NHL diffuse large B, non-Hodgkin's lymphoma (NHL), thymoma, skin malignant melanoma, skin basolioma, skin squamous cell cancer, skin merkel zell cancer, skin benign nevus, lipoma, endometriod carcinoma, endometrium serous carcenoma, small intestine adenocarcinoma, stomach diffuse adenocarcinoma, kidney chromophobic carcinoma, kidney clear cell carcinoma, kidney oncocytoma, kidney papillary carcinoma, Hodgkin lymphoma or liposarcoma.

21. The method of claim 1, wherein said Pin1-associated state is associated with the overexpression of Pin1 and/or DNA damage.

22. The method of claim 1, wherein said Pin1-associated state is associated with an oncogenic protein.

23. The method of claim 1, wherein said Pin1-associated state is associated with Ha-Ras.

24. The method of claim 1, wherein said Pin1-modulating compound has a characteristic inhibition profile (CIP) and has a cytotoxicity effective to treat said Pin1-associated state.

25. The method of claim 24, wherein said Pin1-modulating compound has an IC₅₀ value of less than about 40.

26. The method of claim 25, wherein said IC₅₀ value of between about 10 and about 40.

27. The method of claim 25, wherein said IC₅₀ value of between about 1 and about 10.

28. The method of claim 25, wherein said IC₅₀ value of less than about 1.

29. The method of claim 25, wherein said Pin1-modulating compound has a cytotoxicity of about 3 μM or less as measured by the CBCA.

30. The method of claim 29, wherein said Pin1-modulating compound has a cytotoxicity of about 1.5 μM or less as measured by the CBCA.

31. The method of claim 30, wherein said Pin1-modulating compound has a cytotoxicity of about 1 μM or less as measured by the CBCA.

32. A method for treating cyclin D1 overexpression in a subject comprising administering to said subject an effective amount of a Pin1-modulating compound of formula (I): 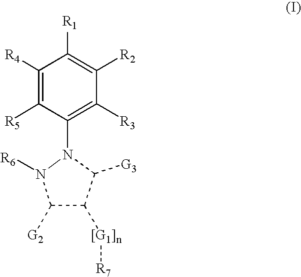
wherein
the dashed lines indicate a single or a double bond;
n is 0, 1, or 2;
G₁ is CH₂, CH, or N;
G₂ and G₃ are independently selected from the group consisting of O, OR₂, NR₂, NR₂R₆, hydrogen, alkyl, aryl;
R₁, R₂, R₃, R₄, R₅, R₆ and R₇ are each independently substituted or unsubstituted: alkyl, alkenyl, alkynyl, aryl, hydrogen, acyl, or any combination thereof;
such that said cyclin D1 overexpression is treated.

33. The method of claim 32, wherein G₂ is O, hydrogen, CH₃, thiophene, or nitrophenyl; G₃ is O, H, or OH; and R₆ is H or CH₃.

34. The method of claim 32, wherein R₇ is a phenyl, a pyridine, an indole, an indene, a pyrazole, a benzoimidazole, a thiophene, a naphthalene, a morpholine, a pyrrolidine, a piperidine, a furan, a tetrahydrofuran, a benzo[1,3]dioxole, a pyrole, a cyclohexene, a furazan-2-oxide, a thioxo-thiazolidinone, an isobenzofuranone, a benzo[4,5]imidazo[1,2-a]pyridine, an isobutyl, a derivative thereof, or
a combination thereof, wherein the combination may further comprise alkyl, alkenyl, alkynyl, or acyl groups.

35. The method of claim 32, wherein R₇ substituted with substituents selected from the group consisting of H, O, OH, Cl, Br, F, I, OEt, OMe, CO₂H, propenyloxy, acetyl, isopropyl, butyloxy, benzyloxy, propyloxy, morpholino, dimethylamino, NO₂, sulfonamide, CO₂CH₂CH₃, —OCH₂CO₂CH₂CH₃, benzene sulfonate, acetamide, Me, t-butyl, propargyl, naphthyl, naphthyloxy, propargyloxy, hexyloxy, octyloxy, dipropylamino, ethylmethylamino, propyloxy, iso-propyl, benzyl, phenyl, methylsulfanyl, phenylsulfanyl, naphthylsulfanyl, benzoyl, , CO₂CH₃, —OCH₂CO₂CH₃, —OCH₂CO₂H, —OCH₂CH₂O—R₃, —CH₂CH₂O—R₃, —OCH₂CH₂S—R₃, derivatives thereof, and combinations thereof.

36. The method of claim 35, wherein R₁, R₂, R₃, R₄, and R₅ are each independently selected from the group consisting of CH₃, Br, I, Cl, H, F, CO₂CH₂CH₃, CF₃, Et, combinations thereof, and derivatives thereof.

37. The method of claim 32, wherein the cyclin D1 overexpression results in neoplastic transformation.

38. The method of claim 32, wherein the cyclin D1 overexpression results in tumor growth.

39. The method of claim 32, wherein said method for treating cyclin D1 overexpression comprises inhibiting tumor growth.

40. The method of claim 32, wherein said method for treating cyclin D1 overexpression comprises preventing the occurrence of tumor growth in the subject.

41. The method of claim 32, wherein said method for treating cyclin D1 overexpression comprises reducing the growth of a pre-existing tumor in the subject.

42. The method of claim 32, wherein the cyclin D1 overexpression results in colon cancer or breast cancer.

43. The method of claim 32, wherein the cyclin D1 overexpression results in a sarcoma or a malignant lymphoma.

44. The method of claim 32, wherein the cyclin D1 overexpression results in esophageal cancer, oligodendroglioma, astrocytoma, glioblastomamultiforme, cervical carcinoma, ovary endometroid cancer, ovary Brenner tumor, ovary mucinous cancer, ovary serous cancer, uterus carcinosarcoma, breast lobular cancer, breast ductal cancer, breast medullary cancer, breast mucinous cancer, breast tubular cancer, thyroid adenocarcinoma, or thyroid follicular cancer.

45. The method of claim 32, wherein the cyclin D1 overexpression results in thyroid medullary cancer, thyroid papillary carcinoma, parathyroid adenocarcinoma, adrenal gland adenoma, adrenal gland cancer, pheochromocytoma, colon adenoma mild displasia, colon adenoma moderate displasia, colon adenoma severe displasia, or colon adenocarcinoma.

46. The method of claim 32, wherein the cyclin D1 overexpression results in esophagus adenocarcinoma, hepatocelluar carcinoma, mouth cancer, gall bladder adenocarcinoma, pancreatic adenocarcinoma, prostate, prostate cancer, testis non-seminomatous cancer, testis seminoma, urinary bladder transitional carcinoma, lung adenocarcinoma, lung large cell cancer, lung small cell cancer, lung squamous cell carcinoma, MALT lymphoma, NHL diffuse large B, non-Hodgkin's lymphoma (NHL), thymoma, skin malignant melanoma, skin basolioma, skin squamous cell cancer, skin merkel zell cancer, skin benign nevus, lipoma, endometriod carcinoma, endometrium serous carcenoma, small intestine adenocarcinoma, stomach diffuse adenocarcinoma, kidney chromophobic carcinoma, kidney clear cell carcinoma, kidney oncocytoma, kidney papillary carcinoma, Hodgkin lymphoma, or a liposarcoma.

47. The method of claim 32, wherein the cyclin D1 overexpression is caused by overexpression of Pin1.

48. The method of claim 32, wherein the cyclin D1 overexpression is caused by DNA damage.

49. The method of claim 32, wherein the cyclin D1 overexpression is caused by an oncogenic protein.

50. The method of claim 32, wherein cyclin D1 overexpression is caused by Ha-Ras.

51. The method of claim 32, wherein said Pin1 modulating compound is a Pin1 inhibiting compound.

52. The method of claim 32, wherein said compound is selected from the group consisting of compounds listed in Table 1, and derivatives thereof.

53. The method of claim 32-50, wherein said compound is selected from the group consisting of compounds listed in Table 2, and derivatives thereof.

54. The method of claim 32, wherein said Pin1-modulating compound has a characteristic inhibition profile (CIP) and has a cytotoxicity effective to treat said Pin1-associated state.

55. The method of claim 54, wherein said Pin1-modulating compound has an IC₅₀ value of less than about 40.

56. The method of claim 55, wherein said IC₅₀ value of between about 10 and about 40.

57. The method of claim 55, wherein said IC₅₀ value of between about 1 and about 10.

58. The method of claim 55, wherein said IC₅₀ value of less than about 1.

59. The method of claim 54, wherein said Pin1-modulating compound has a cytotoxicity of about 3 μM or less as measured by the CBCA.

60. The method of claim 59, wherein said Pin1-modulating compound has a cytotoxicity of about 1.5 μM or less as measured by the CBCA.

61. The method of claim 60, wherein said Pin1-modulating compound has a cytotoxicity of about 1 μM or less as measured by the CBCA.

62. A packaged Pin1-associated state treatment, comprising a Pin1-modulating compound of formula (I): 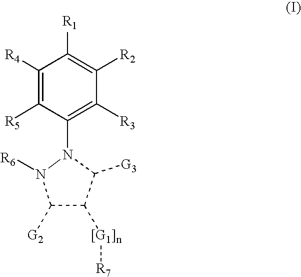
wherein
the dashed lines indicate a single or a double bond;
n is 0, 1, or 2;
G₁ is CH₂, CH, or N;
G₂ and G₃ are independently selected from the group consisting of O, OR₂, NR₂, NR₂R₆, hydrogen, alkyl, aryl;
R₁, R₂, R₃, R₄, R₅, R₆ and R₇ are each independently substituted or unsubstituted: alkyl, alkenyl, alkynyl, aryl, hydrogen, acyl, or any combination thereof;
packaged with instructions for using an effective amount of the Pin1-modulating compound to treat a Pin1-associated state.

63. The packaged Pin1-associated state treatment of claim 62, wherein G₂ is O, hydrogen, CH₃, thiophene, or nitrophenyl; G₃ is O, H, or OH; and R₆ is H or CH₃.

64. The packaged Pin1-associated state treatment of claim 62, wherein R₇ is a phenyl, a pyridine, an indole, an indene, a pyrazole, a benzoimidazole, a thiophene, a naphthalene, a morpholine, a pyrrolidine, a piperidine, a furan, a tetrahydrofuran, a benzo[1,3]dioxole, a pyrole, a cyclohexene, a furazan-2-oxide, a thioxo-thiazolidinone, an isobenzofuranone, a benzo[4,5]imidazo[1,2-a]pyridine, an isobutyl, a derivative thereof, or
a combination thereof, wherein the combination may further comprise alkyl, alkenyl, alkynyl, or acyl groups.

65. The packaged Pin1-associated state treatment of claim 62, wherein R₇ substituted with substituents selected from the group consisting of H, O, OH, Cl, Br, F, I, OEt, OMe, CO₂H, propenyloxy, acetyl, isopropyl, butyloxy, benzyloxy, propyloxy, morpholino, dimethylamino, NO₂, sulfonamide, CO₂CH₂CH₃, —OCH₂CO₂CH₂CH₃, benzene sulfonate, acetamide, Me, t-butyl, propargyl, naphthyl, naphthyloxy, propargyloxy, hexyloxy, octyloxy, dipropylamino, ethylmethylamino, propyloxy, iso-propyl, benzyl, phenyl, methylsulfanyl, phenylsulfanyl, naphthylsulfanyl, benzoyl, , CO₂CH₃, —OCH₂CO₂CH₃, —OCH₂CO₂H, —OCH₂CH₂O—R₃, —CH₂CH₂O—R₃, —OCH₂CH₂S—R₃, derivatives thereof, and combinations thereof.

66. The packaged Pin1-associated state treatment of claim 65, wherein R₁, R₂, R₃, R₄, and R₅ are each independently selected from the group consisting of CH₃, Br, I, Cl, H, F, CO₂CH₂CH₃, CF₃, Et, combinations thereof, and derivatives thereof.

67. The packaged Pin1-associated state treatment of claim 62, wherein said Pin1 modulating compound is a Pin1 inhibiting compound.

68. The packaged Pin1-associated state treatment of claim 62, wherein said compound is selected from the group consisting of compounds listed in Table 1, and derivatives thereof.

69. The packaged Pin1-associated state treatment of claim 62, wherein said compound is selected from the group consisting of compounds listed in Table 2, and derivatives thereof.

70. A packaged cyclin D1 overexpression treatment, comprising a Pin1-modulating compound of formula (I): 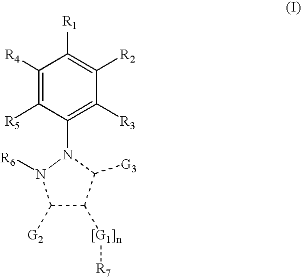
wherein
the dashed lines indicate a single or a double bond;
n is 0, 1, or 2;
G₁ is CH₂, CH, or N;
G₂ and G₃ are independently selected from the group consisting of O, OR₂, NR₂, NR₂R₆, hydrogen, alkyl, aryl;
R₁, R₂, R₃, R₄, R₅, R₆ and R₇ are each independently substituted or unsubstituted: alkyl, alkenyl, alkynyl, aryl, hydrogen, acyl, or any combination thereof;
packaged with instructions for using an effective amount of the Pin1-modulating compound to treat cyclin D1 overexpression.

71. The packaged cyclin D1 overexpression treatment of claim 70, wherein G₂ is O, hydrogen, CH₃, thiophene, or nitrophenyl; G₃ is O, H, or OH; and R₆ is H or CH₃.

72. The packaged cyclin D1 overexpression treatment of claim 70, wherein R₇ is a phenyl, a pyridine, an indole, an indene, a pyrazole, a benzoimidazole, a thiophene, a naphthalene, a morpholine, a pyrrolidine, a piperidine, a furan, a tetrahydrofuran, a benzo[1,3]dioxole, a pyrole, a cyclohexene, a furazan-2-oxide, a thioxo-thiazolidinone, an isobenzofuranone, a benzo[4,5]imidazo[1,2-a]pyridine, an isobutyl, a derivative thereof, or
a combination thereof, wherein the combination may further comprise alkyl, alkenyl, alkynyl, or acyl groups.

73. The packaged cyclin D1 overexpression treatment of claim 70, wherein R₇ substituted with substituents selected from the group consisting of H, O, OH, Cl, Br, F, I, OEt, OMe, CO₂H, propenyloxy, acetyl, isopropyl, butyloxy, benzyloxy, propyloxy, morpholino, dimethylamino, NO₂, sulfonamide, CO₂CH₂CH₃, —OCH₂CO₂CH₂CH₃, benzene sulfonate, acetamide, Me, t-butyl, propargyl, naphthyl, naphthyloxy, propargyloxy, hexyloxy, octyloxy, dipropylamino, ethylmethylamino, propyloxy, iso-propyl, benzyl, phenyl, methylsulfanyl, phenylsulfanyl, naphthylsulfanyl, benzoyl,, CO₂CH₃, —OCH₂CO₂CH₃, —OCH₂CO₂H, —OCH₂CH₂O—R₃, —CH₂CH₂O—R₃, —OCH₂CH₂S—R₃, derivatives thereof, and combinations thereof.

74. The packaged cyclin D1 overexpression treatment of claim 73, wherein R₁, R₂, R₃, R₄, and R₅ are each independently selected from the group consisting of CH₃, Br, I, Cl, H, F, CO₂CH₂CH₃, CF₃, Et, combinations thereof, and derivatives thereof.

75. The packaged cyclin D1 overexpression treatment Pin1-associated state treatment of claim 70, wherein said Pin1 modulating compound is a Pin1 inhibiting compound.

76. The packaged cyclin D1 overexpression treatment Pin1-associated state treatment of claim 70, wherein said compound is selected from the group consisting of compounds listed in Table 1, and derivatives thereof.

77. The packaged cyclin D1 overexpression treatment Pin1-associated state treatment of claim 70, wherein said compound is selected from the group consisting of compounds listed in Table 2, and derivatives thereof.

78. A packaged cancer treatment, comprising a Pin1-modulating compound of formula (I): 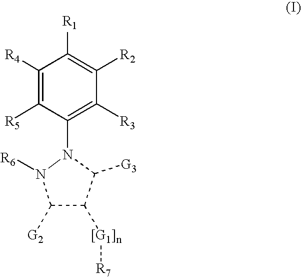
wherein
the dashed lines indicate a single or a double bond;
n is 0, 1, or 2;
G₁ is CH₂, CH, or N;
G₂ and G₃ are independently selected from the group consisting of O, OR₂, NR₂, NR₂R₆, hydrogen, alkyl, aryl;
R₁, R₂, R₃, R₄, R₅, R₆ and R₇ are each independently substituted or unsubstituted: alkyl, alkenyl, alkynyl, aryl, hydrogen, acyl, or any combination thereof;
packaged with instructions for using an effective amount of the Pin1-modulating compound to treat cancer.

79. The packaged cancer treatment of claim 78, wherein G₂ is O, hydrogen, CH₃, thiophene, or nitrophenyl; G₃ is O, H, or OH; and R₆ is H or CH₃.

80. The packaged cancer treatment of claim 78, wherein R₇ is a phenyl, a pyridine, an indole, an indene, a pyrazole, a benzoimidazole, a thiophene, a naphthalene, a morpholine, a pyrrolidine, a piperidine, a furan, a tetrahydrofuran, a benzo[1,3]dioxole, a pyrole, a cyclohexene, a furazan-2-oxide, a thioxo-thiazolidinone, an isobenzofuranone, a benzo[4,5]imidazo[1,2-a]pyridine, an isobutyl, a derivative thereof, or
a combination thereof, wherein the combination may further comprise alkyl, alkenyl, alkynyl, or acyl groups.

81. The packaged cancer treatment of claim 78, wherein R₇ substituted with substituents selected from the group consisting of H, O, OH, Cl, Br, F, I, OEt, OMe, CO₂H, propenyloxy, acetyl, isopropyl, butyloxy, benzyloxy, propyloxy, morpholino, dimethylamino, NO₂, sulfonamide, CO₂CH₂CH₃, —OCH₂CO₂CH₂CH₃, benzene sulfonate, acetamide, Me, t-butyl, propargyl, naphthyl, naphthyloxy, propargyloxy, hexyloxy, octyloxy, dipropylamino, ethylmethylamino, propyloxy, iso-propyl, benzyl, phenyl, methylsulfanyl, phenylsulfanyl, naphthylsulfanyl, benzoyl,, CO₂CH₃, —OCH₂CO₂CH₃, —OCH₂CO₂H, —OCH₂CH₂O—R₃, —CH₂CH₂O—R₃, —OCH₂CH₂S—R₃, derivatives thereof, and combinations thereof.

82. The packaged cancer treatment of claim 81, wherein R₁, R₂, R₃, R₄, and R₅ are each independently selected from the group consisting of CH₃, Br, I, Cl, H, F, CO₂CH₂CH₃, CF₃, Et, combinations thereof, and derivatives thereof.

83. The packaged cancer treatment of claim 78, wherein said Pin1 modulating compound is a Pin1 inhibiting compound.

84. The packaged cancer treatment of claim 78, wherein said compound is selected from the group consisting of compounds listed in Table 1, and derivatives thereof.

85. The packaged cancer treatment of claim 78, wherein said compound is selected from the group consisting of compounds listed in Table 2, and derivatives thereof.

86. A method for treating a Pin1-associated state in a subject comprising administering to a subject an effective amount of a combination of a Pin1-modulating compound of formula (I): 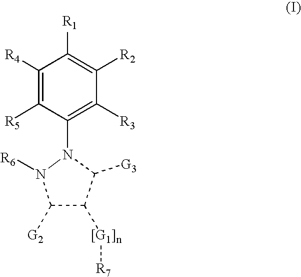
wherein
the dashed lines indicate a single or a double bond;
n is 0, 1, or 2;
G₁ is CH₂, CH, or N;
G₂ and G₃ are independently selected from the group consisting of O, OR₂, NR₂, NR₂R₆, hydrogen, alkyl, aryl;
R₁, R₂, R₃, R₄, R₅, R₆ and R₇ are each independently substituted or unsubstituted: alkyl, alkenyl, alkynyl, aryl, hydrogen, acyl, or any combination thereof;
a hyperplastic inhibitory agent such that the Pin1-associated state is treated.

87. The method of claim 86, wherein G₂ is O, hydrogen, CH₃, thiophene, or nitrophenyl; G₃ is O, H, or OH; and R₆ is H or CH₃.

88. The method of claim 86, wherein R₇ is a phenyl, a pyridine, an indole, an indene, a pyrazole, a benzoimidazole, a thiophene, a naphthalene, a morpholine, a pyrrolidine, a piperidine, a furan, a tetrahydrofuran, a benzo[1,3]dioxole, a pyrole, a cyclohexene, a furazan-2-oxide, a thioxo-thiazolidinone, an isobenzofuranone, a benzo[4,5]imidazo[1,2-a]pyridine, an isobutyl, a derivative thereof, or
a combination thereof, wherein the combination may further comprise alkyl, alkenyl, alkynyl, or acyl groups.

89. The method of claim 86, wherein R₇ substituted with substituents selected from the group consisting of H, O, OH, Cl, Br, F, I, OEt, OMe, CO₂H, propenyloxy, acetyl, isopropyl, butyloxy, benzyloxy, propyloxy, morpholino, dimethylamino, NO₂, sulfonamide, CO₂CH₂CH₃, —OCH₂CO₂CH₂CH₃, benzene sulfonate, acetamide, Me, t-butyl, propargyl, naphthyl, naphthyloxy, propargyloxy, hexyloxy, octyloxy, dipropylamino, ethylmethylamino, propyloxy, iso-propyl, benzyl, phenyl, methylsulfanyl, phenylsulfanyl, naphthylsulfanyl, benzoyl, , CO₂CH₃, —OCH₂CO₂CH₃, —OCH₂CO₂H, —OCH₂CH₂O—R₃, —CH₂CH₂O—R₃, —OCH₂CH₂S—R₃, derivatives thereof, and combinations thereof.

90. The method of claim 89, wherein R₁, R₂, R₃, R₄, and R₅ are each independently selected from the group consisting of CH₃, Br, I, Cl, H, F, CO₂CH₂CH₃, CF₃, Et, combinations thereof, and derivatives thereof.

91. The method of claim 86, wherein said Pin1 modulating compound is a Pin1 inhibiting compound.

92. The method of claim 86, wherein said compound is selected from the group consisting of compounds listed in Table 1, and derivatives thereof.

93. The method of claim 86, wherein said compound is selected from the group consisting of compounds listed in Table 2, and derivatives thereof.

94. The method of claim 86, wherein said Pin1-modulating compound has a characteristic inhibition profile (CIP) and has a cytotoxicity effective to treat said Pin1-associated state.

95. The method of claim 94, wherein said Pin1-modulating compound has an IC₅₀ value of less than about 40.

96. The method of claim 95, wherein said IC₅₀ value of between about 10 and about 40.

97. The method of claim 95, wherein said IC₅₀value of between about 1 and about 10.

98. The method of claim 95, wherein said IC₅₀ value of less than about 1.

99. The method of claim 94, wherein said Pin1-modulating compound has a cytotoxicity of 3 μM or less as measured by the CBCA.

100. The method of claim 99, wherein said Pin1-modulating compound has a cytotoxicity of 1.5 μM or less as measured by the CBCA.

101. The method of claim 100, wherein said Pin1-modulating compound has a cytotoxicity of 1 μM or less as measured by the CBCA.

102. The method of claim 86, wherein the hyperplastic inhibitory agent is tamoxifen.

103. The method of claim 86, wherein the hyperplastic inhibitory agent is paclitaxel.

104. The method of claim 86, wherein the hyperplastic inhibitory agent is docetaxel.

105. The method of claim 86, wherein the hyperplastic inhibitory agent is interleukin-2.

106. The method of claim 86, wherein the hyperplastic inhibitory agent is rituximab.

107. The method of claim 86, wherein the hyperplastic inhibitory agent is tretinoin.

108. The method of claim 86, wherein the hyperplastic inhibitory agent is methotrexate.

109. The method of claim 86, wherein the hyperplastic inhibitory agent is a radiation therapy treatment.

110. A method for treating cancer in a subject comprising administering to a subject an effective amount of a combination of a Pin1-modulating compound of formula (I): 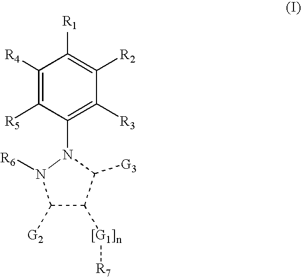
wherein
the dashed lines indicate a single or a double bond;
n is 0, 1, or 2;
G₁ is CH₂, CH, or N;
G₂ and G₃ are independently selected from the group consisting of O, OR₂, NR₂, NR₂R₆, hydrogen, alkyl, aryl;
R₁, R₂, R₃, R₄, R₅, R₆ and R₇ are each independently substituted or unsubstituted: alkyl, alkenyl, alkynyl, aryl, hydrogen, acyl, or any combination thereof;
a hyperplastic inhibitory agent such that the cancer is treated.

111. The method of claim 110, wherein G₂ is O, hydrogen, CH₃, thiophene, or nitrophenyl; G₃ is O, H, or OH; and R₆ is H or CH₃.

112. The method of claim 110, wherein R₇ is a phenyl, a pyridine, an indole, an indene, a pyrazole, a benzoimidazole, a thiophene, a naphthalene, a morpholine, a pyrrolidine, a piperidine, a furan, a tetrahydrofuran, a benzo[1,3]dioxole, a pyrole, a cyclohexene, a furazan-2-oxide, a thioxo-thiazolidinione, an isobenzofuranone, a benzo[4,5]imidazo[1,2-a]pyridine, an isobutyl, a derivative thereof, or
a combination thereof, wherein the combination may further comprise alkyl, alkenyl, alkynyl, or acyl groups.

113. The method of claim 110, wherein R₇ substituted with substituents selected from the group consisting of H, O, OH, Cl, Br, F, I, OEt, OMe, CO₂H, propenyloxy, acetyl, isopropyl, butyloxy, benzyloxy, propyloxy, morpholino, dimethylamino, NO₂, sulfonamide, CO₂CH₂CH₃, —OCH₂CO₂CH₂CH₃, benzene sulfonate, acetamide, Me, t-butyl, propargyl, naphthyl, naphthyloxy, propargyloxy, hexyloxy, octyloxy, dipropylamino, ethylmethylamino, propyloxy, iso-propyl, benzyl, phenyl, methylsulfanyl, phenylsulfanyl, naphthylsulfanyl, benzoyl, , CO₂CH₃, —OCH₂CO₂CH₃, —OCH₂CO₂H, —OCH₂CH₂O—R₃, —CH₂CH₂O—R₃, —OCH₂CH₂S—R₃, derivatives thereof, and combinations thereof.

114. The method of claim 113, wherein R₁, R₂, R₃, R₄, and R₅ are each independently selected from the group consisting of CH₃, Br, I, Cl, H, F, CO₂CH₂CH₃, CF₃, Et, combinations thereof, and derivatives thereof.

115. The method of claim 110, wherein said Pin1 modulating compound is a Pin1 inhibiting compound.

116. The method of claim 110, wherein said compound is selected from the group consisting of compounds listed in Table 1, and derivatives thereof.

117. The method of claim 110, wherein said compound is selected from the group consisting of compounds listed in Table 2, and derivatives thereof.

118. A method for treating cyclin D1 overexpression in a subject comprising administering to a subject an effective amount of a combination of a Pin1-modulating compound of formula (I): 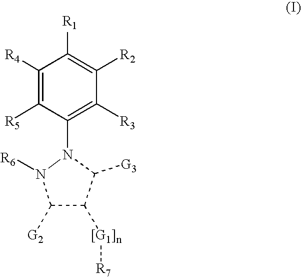
wherein
the dashed lines indicate a single or a double bond;
n is 0, 1, or 2;
G₁ is CH₂, CH, or N;
G₂ and G₃ are independently selected from the group consisting of O, OR₂, NR₂, NR₂R₆, hydrogen, alkyl, aryl;
R₁, R₂, R₃, R₄, R₅, R₆ and R₇ are each independently substituted or unsubstituted: alkyl, alkenyl, alkynyl, aryl, hydrogen, acyl, or any combination thereof;
a hyperplastic inhibitory agent such that the cyclin D1 overexpression is treated.

119. The method of claim 118, wherein G₂ is O, hydrogen, CH₃, thiophene, or nitrophenyl; G₃ is O, H, or OH; and R₆ is H or CH₃.

120. The method of claim 118, wherein R₇ is a phenyl, a pyridine, an indole, an indene, a pyrazole, a benzoimidazole, a thiophene, a naphthalene, a morpholine, a pyrrolidine, a piperidine, a furan, a tetrahydrofuran, a benzo[1,3]dioxole, a pyrole, a cyclohexene, a furazan-2-oxide, a thioxo-thiazolidinone, an isobenzofuranone, a benzo[4,5]imidazo[1,2-a]pyridine, an isobutyl, a derivative thereof, or a combination thereof, wherein the combination may further comprise alkyl, alkenyl, alkynyl, or acyl groups.

121. The method of claim 118, wherein R₇ substituted with substituents selected from the group consisting of H, O, OH, Cl, Br, F, I, OEt, OMe, CO₂H, propenyloxy, acetyl, isopropyl, butyloxy, benzyloxy, propyloxy, morpholino, dimethylamino, NO₂, sulfonamide, CO₂CH₂CH₃, —OCH₂CO₂CH₂CH₃, benzene sulfonate, acetamide, Me, t-butyl, propargyl, naphthyl, naphthyloxy, propargyloxy, hexyloxy, octyloxy, dipropylamino, ethylmethylamino, propyloxy, iso-propyl, benzyl, phenyl, methylsulfanyl, phenylsulfanyl, naphthylsulfanyl, benzoyl, , CO₂CH₃, —OCH₂CO₂CH₃, —OCH₂CO₂H, —OCH₂CH₂O—R₃, —CH₂CH₂O—R₃, —OCH₂CH₂S—R₃, derivatives thereof, and combinations thereof.

122. The method of claim 121, wherein R₁, R₂, R₃, R₄, and R₅ are each independently selected from the group consisting of CH₃, Br, I, Cl, H, F, CO₂CH₂CH₃, CF₃, Et, combinations thereof, and derivatives thereof.

123. The method of claim 118, wherein said Pin1 modulating compound is a Pin1 inhibiting compound.

124. The method of claim 118, wherein said compound is selected from the group consisting of compounds listed in Table 1, and derivatives thereof.

125. The method of claim 118, wherein said compound is selected from the group consisting of compounds listed in Table 2, and derivatives thereof.

126. A Pin1-modulator comprising formula (I): 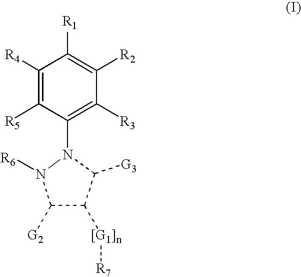
wherein
the dashed lines indicate a single or a double bond;
n is 0, 1, or 2;
G₁ is CH₂, CH, or N;
G₂ and G₃ are independently selected from the group consisting of O, OR₂, NR₂, NR₂R₆, hydrogen, alkyl, aryl; and
R₁, R₂, R₃, R₄, R₅, R₆ and R₇ are each independently substituted or unsubstituted: alkyl, alkenyl, alkynyl, aryl, hydrogen, acyl, or any combination thereof.

127. The Pin1-modulator of claim 126, wherein G₂ is O, hydrogen, CH₃, thiophene, or nitrophenyl; G₃ is O, H, or OH; and R₆ is H or CH₃.

128. The Pin1-modulator of claim 126, wherein R₇ is a phenyl, a pyridine, an indole, an indene, a pyrazole, a benzoimidazole, a thiophene, a naphthalene, a morpholine, a pyrrolidine, a piperidine, a furan, a tetrahydrofuran, a benzo[1,3]dioxole, a pyrole, a cyclohexene, a furazan-2-oxide, a thioxo-thiazolidinone, an isobenzofuranone, a benzo[4,5]imidazo[1,2-a]pyridine, an isobutyl, a derivative thereof, or
a combination thereof, wherein the combination may further comprise alkyl, alkenyl, alkynyl, or acyl groups.

129. The Pin1-modulator of claim 126, wherein R₇ substituted with substituents selected from the group consisting of H, O, OH, Cl, Br, F, I, OEt, OMe, CO₂H, propenyloxy, acetyl, isopropyl, butyloxy, benzyloxy, propyloxy, morpholino, dimethylamino, NO₂, sulfonamide, CO₂CH₂CH₃, —OCH₂CO₂CH₂CH₃, benzene sulfonate, acetamide, Me, t-butyl, propargyl, naphthyl, naphthyloxy, propargyloxy, hexyloxy, octyloxy, dipropylamino, ethylmethylamino, propyloxy, iso-propyl, benzyl, phenyl, methylsulfanyl, phenylsulfanyl, naphthylsulfanyl, benzoyl, , CO₂CH₃, —OCH₂CO₂CH₃, —OCH₂CO₂H, —OCH₂CH₂O—R₃, —CH₂CH₂O—R₃, —OCH₂CH₂S—R₃, derivatives thereof, and combinations thereof.

130. The Pin1-modulator of claim 129, wherein R₁, R₂, R₃, R₄, and R₅ are each independently selected from the group consisting of CH₃, Br, I, Cl, H, F, CO₂CH₂CH₃, CF₃, Et, combinations thereof, and derivatives thereof.

131. The Pin1-modulator of claim 126, wherein said Pin1 modulating compound is a Pin1 inhibiting compound.

132. The Pin1-modulator of claim 126, wherein said compound is selected from the group consisting of compounds listed in Table 1, and derivatives thereof.

133. The Pin1-modulator of claim 126, wherein said compound is selected from the group consisting of compounds listed in Table 2, and derivatives thereof.

134. A pharmaceutical composition comprising a Pin1-modulating compound of claim 1, 32, 86, 110, 118, or 126, and a pharmaceutically acceptable carrier.

135. The pharmaceutical composition of claim 134, wherein said compound is selected from the group consisting of compounds listed in Table 1, and derivatives thereof.

136. The pharmaceutical composition of claim 134, wherein said compound is selected from the group consisting of compounds listed in Table 2, and derivatives thereof.
